(19) United States
(12) Patent Application Publication
(10) Pub. No.: US 2004/0219563 A1
(43) Pub. Date: Nov. 4, 2004

(54) Method using gene trapped stem cells for making pathways of stem cell differentiation and making and isolating differentiated cells

(76) Inventors: Michael West, Southborough, MA (US); Karen B. Chapman, Southborough, MA (US)

Correspondence Address: MERCHANT & GOULD PC, P.O. BOX 2903, MINNEAPOLIS, MN, 55402-0903, US

(21) Appl. No.: 10/685,693

(22) Filed: Oct. 16, 2003

Related U.S. Application Data
(60) Provisional application No. 60/418,333, filed on Oct. 16, 2002.

(51) Int. Cl. C12Q 1/68; C12N 5/08; C12N 15/85

(52) U.S. Cl. 435/6; 435/455; 435/366

(57) ABSTRACT

Transcriptional assays for identifying genes involved in differentiation are provided. These assays use stem cells into which are randomly integrated marker genes.

### FIELD OF THE INVENTION

The present invention stands in the fields of cell and molecular biology, embryology, and medicine. In particular, the invention provides methods for identifying the nucleotide sequences and temporal and spatial patterns of expression of the genes that control the differentiation of stem cells into all of the partially and fully differentiated cells of a mammal. The invention also involves fixing the information obtained by such methods in tangible form that permits its communication and use in new methods for identifying conditions under which isolated stem cells differentiate ex vivo, for identifying cell surface antigens that facilitate the isolation of those cells, and for inducing isolated stem cells to differentiate ex vivo into specific cell types that are useful for basic research, drug discovery and testing, toxicological studies, animal testing of cell therapy, and for actual cell therapy in human and non-human patients in need of cell therapy.

### BACKGROUND OF THE INVENTION

The present need for histocompatible cells and tissues for transplant

There presently is great need for new sources of cells and tissues for therapeutic transplant that are histocompatible with the transplant recipients. Transplanted cells or tissue are rejected by the immune system of the transplant recipient unless they are histocompatible with the recipient. Rejection occurs as a result of an adaptive immune response to alloantigens or xenoantigens on the grafted tissue by the transplant recipient. The alloantigens or xenoantigens are typically on “non-self” proteins, i.e., antigenic proteins that are identified as foreign by the immune system of a transplant recipient. The proteins on the surfaces of transplanted tissue that most strongly evoke rejection are the antigenic proteins encoded by the MHC (major histocompatibility complex) genes. In order to match the types of MHC molecules present in the transplant tissue with those of a recipient, assays are performed to identify the MHC types present on the cells of tissue to be transplanted, and on the cells of the transplant recipient. The number of people in need of cell, tissue, and organ transplants is far greater than the available supply of cells, tissues, and organs suitable for transplantation; as a result, it is frequently impossible to obtain a good match between a recipient's MHC proteins those of cells or tissue that are available for transplant. Hence, many transplant recipients must wait for an MHC-matched transplant to become available, or accept a transplant that is not MHC-matched. If the latter is necessary, the transplant recipient must rely on heavier doses of immunosuppressive drugs and face a greater risk of rejection than would be the case if MHC matching had been possible. New sources of histocompatible cells and tissues for therapeutic transplant to non-human mammals in need of such transplant are also needed in veterinary medicine.

### Stem Cells as a Source of Cells and Tissues for Therapy

Embryonic stem (ES) cells are undifferentiated stem cells that are derived from the inner cell mass of a blastocyst embryo. ES cells appear to have unlimited proliferative potential, and are capable of differentiating into all of the specialized cell types of a mammal, including the three embryonic germ layers (endoderm, mesoderm, and ectoderm), and all somatic cell lineages and the germ line. For example, ES cells can be induced to differentiate in vitro into cardiomyocytes (Paquin et al., Proc. Nat. Acad. Sci. (2002) 99:9550-9555), hematopoietic cells (Weiss et al., Hematol. Oncol. Clin. N. Amer. (1997) 11(6):1185-98; also U.S. Pat. No. 6,280,718), insulin-secreting beta cells (Assady et al., Diabetes (2001) 50(8):1691-1697), and neural progenitors capable of differentiating into astrocytes, oligodendrocytes, and mature neurons (Reubinoff et al., Nature Biotechnology (2001) 19:1134-1140; also U.S. Pat. No. 5,851,832). According to data from the Centers for Disease Control and Prevention, as many as 3,000 Americans die every day from diseases that in the future may be treatable with tissues derived from ES cells. In addition to generating functional replacement cells such as cardiomyocytes, neurons, or insulin-producing β cells, ES cells may be able to reconstitute more complex tissues and organs, including blood vessels, myocardial “patches,” kidneys, and even entire hearts (Atala, A. & Lanza, R. P. Methods of Tissue Engineering, Academic Press, San Diego, Calif., 2001).

In order to fully realize the potential benefits of producing cells and tissues for transplant from ES cells and other totipotent, nearly totipotent, or pluripotent stem cells, sources of adequate quantities of such stem cells that are histocompatible with those in need of transplants must be found, and methods for directing the stem cells to differentiate into all of the different cells needed, and means for purifying them for transplant, must be obtained.

### Stem Cells Produced by Nuclear Transfer Cloning

Advanced Cell Technology, Inc. (ACT), the assignee of this application, and other groups have developed methods for transferring the genetic information in the nucleus of a somatic or germ cell from a child or adult into an unfertilized egg cell, and culturing the resulting cell to divide and form a blastocyst embryo having the genotype of the somatic or germ nuclear donor cell. Methods for cloning by such methods, referred to as “somatic cell nuclear transfer” because somatic donor cells are commonly used, are described, for example, in U.S. Pat. Nos. 5,994,619, 6,235,969, and 6,252,133, the contents of which are incorporated herein in their entirety. Totipotent ES or ES-like cells derived from the inner cell mass of a blastocyst generated by somatic cell nuclear transfer have the genomic DNA of the somatic nuclear donor cell, and differentiated cells derived from such ES cells are histocompatible with the individual from whom the somatic donor cell was obtained. Hence, one approach to overcoming the shortage of histocompatible cells and tissues suitable for transplant therapies, is to perform nuclear transfer cloning using a somatic donor cell from the human or non-human mammal that is in need of such a transplant, derive ES cells from the resulting blastocysts, and culture the ES cells under conditions that induce or direct their differentiation into cells of the type that are needed for transplant.

Cells and tissues generated by somatic cell nuclear transfer cloning are nearly completely autologous—all of the cells' proteins except those encoded by the cells' mitochondria, which derive from the oocyte, are encoded by the patient's own DNA. Concerns that allogeneic mitochondria in cells obtained by somatic cell nuclear transfer cloning and transplanted into a syngeneic transplant recipient would elicit rejection of the transplant have been allayed by recent studies by researchers at ACT showing that cells and tissues produced by nuclear transfer cloning and transplanted into syngeneic cattle do not elicit rejection. Tissue-engineered constructs comprising three different differentiated bovine cell types generated by bovine somatic nuclear transplant cloning were transplanted into the syngeneic cattle, where they survived and grew for 12 weeks without rejection, while allogeneic control cells were rejected. See Lanza et al. (Nature Biotechnology, 2002, 20:689-695), the contents of which are incorporated herein in their entirety. Cells and tissues produced by somatic cell nuclear transfer cloning can thus be therapeutically grafted or transplanted to a syngeneic individual without triggering the severe rejection response that results when foreign cells or tissue are transplanted. Recipients of syngeneic cell and tissue transplants produced by somatic cell nuclear transfer cloning therefore do not need to be exposed to the risk of serious and potentially life-threatening complications that are associated with the use of immunosuppressive drugs and/or immunomodulatory protocols to prevent rejection of allogeneic transplants.

Methods that use nuclear transfer cloning to produce cells and tissues for transplant therapies that are histocompatible with the transplant recipients are described in co-owned and co-pending U.S. application Ser. No. 09/797,684 filed Mar. 5, 2001, which further describes assay methods for determining the immune-compatibility of cells and tissues for transplant; PCT application No. 10/112,939 filed Apr. 2, 2002, which also describes methods for inducing stem cells to differentiate into cell types useful for transplant therapy; and U.S. Application PCT/US02/26945, filed Aug. 26, 2002 which claims priority to U.S. Provisional Application No. 60/314,316 filed Aug. 24, 2001, which also describes methods for screening to identify conditions inducing stem cells to differentiate into cell types useful for transplant therapy. Such methods are also described in co-owned and co-pending U.S. application Ser. No. 09/995,659 filed Nov. 29, 2001, and International Application No. PCT/US02/22857 filed Jul. 18, 2002, which further describe methods for producing histocompatible cells and tissues for transplant by androgenesis and gynogenesis, and U.S. application Ser. No. 09/520,879 filed Apr. 5, 2000, which further describes methods for producing “rejuvenated” or “hyper-young” cells having increased proliferative potential relative to cells of the donor animal. Such methods are also described in co-owned and co-pending U.S. application Ser. Nos. 10/228,296 and 10/228,316, both filed on Aug. 27, 2002, which further describe methods for making histocompatible cells and tissues for transplant by trans-differentiation and de-differentiation, respectively, of differentiated somatic cells. The disclosures of all of the above-listed applications are incorporated herein by reference in their entirety.

A bank of ES cells with homozygous MHC alleles for cell transplant therapies

As an alternative to using nuclear transfer cloning to produce syngeneic ES cells de novo and inducing these to differentiate into the required cells for every patient that is in need of therapeutic transplant, nuclear transfer cloning can be used to prepare a bank of pre-made ES cell lines, each of which is homozygous for at least one MHC gene. The MHC genes, in the case of humans also referred to as HLA (human leukocyte antigen) genes or alleles, are highly polymorphic, and a bank of different ES cell lines that includes an ES cell line that is homozygous for each of the variants of the MHC alleles present in the human population will include a large number of different ES cell lines. Once a bank of such ES cells having homozygous MHC alleles is produced, it will be possible to provide a patient in need of cell transplant with MHC-matched cells and tissues by selecting and expanding a line of ES cells from the ES cell bank that has MHC allele(s) that match one of those of the patient, and inducing the ES cells to differentiate into the type of cells that the patient requires. Methods for preparing a bank of ES cell lines that are homozygous for the MHC alleles, and for using these to provide MHC-matched cells and tissues for transplantation therapies are described in the co-pending U.S. Patent Application entitled, “A Bank of Nuclear Transfer-Generated Stem Cells for Transplantation Having Homozygous MHC Alleles, and Methods for Making and Using Such a Stem Cell Bank, filed May 24, 2002, the disclosure of which is incorporated herein by reference in its entirety.

### Directed Differentiation of Stem Cells

Totipotent and nearly totipotent embryo-derived stem cells can be induced to differentiate into a wide variety of cell types, many of which are needed for cell transplantation therapies. Efforts to study the factors that regulate and control the differentiation of stem cells, particularly ES cells, into different, specific cell types, have generally followed either of two distinct approaches.

### Identification of Conditions that Induce Stem Cells to Differentiate

One approach taken has been to expose the stem cells to one or a few different solutions containing a relatively small number of growth factors or cytokines, and monitoring to see if the stem cells differentiate to acquire a morphology and/or to express a marker gene that is characteristic of a specific cell type. For example, Anderson et al. demonstrated that inner cell masses (ICM) and embryonic discs from bovine and porcine blastocysts will develop into teratomas containing differentiated cell types from ectodermal, mesodermal and endodermal origins when transplanted under the kidney capsule of athymic mice (see Animal Repro. Sci. (1996) 45: 231-240). Thomson et al. describe transplanting primate ES cells into muscles of immunodeficient mice to generate teratomas that contain cells of the three embryonic germ layers, including tissues resembling neural tube, embryonic ganglia, neurons, and astrocytes (APMIS (1998) 106(1):149-156). Cells of all three germ layers, including neural precursor cells that test positive for nestin, an intermediate filament protein produced in the developing central nervous system and widely used as a marker for proliferating neural progenitor cells in the nervous system, have been identified in embryoid bodies grown from ES cells of mice (Lee et al., Nature Biotech. (2000) 18:675-679), cynomologous monkeys (Macaca fascicularis) (Cibelli et al., Science (2002) 295:819), and humans (Zhang et al., Nature Biotech. (2001) 19:1129-1133) cultured in vitro. Zhang et al. also reported culturing nestin-positive neural stem cells isolated from human embryoid bodies under conditions that induce their differentiation into the three major cell types of the central nervous system (p. 1130).

The foregoing reports describe the derivation of precursor or differentiated cells that appear to arise randomly or spontaneously in embryoid bodies and teratomas generated from totipotent ES and EG cells. Presently, there is also strong interest in identifying chemical, biological, and physical agents or conditions that induce totipotent or nearly totipotent cells such as ES and EG cells to differentiate directly into the desired differentiated cells ex vivo, in order to develop efficient methods for producing characterized populations of differentiated cells that are useful for cell therapy. Field has described plating murine ES cells onto uncoated petri dishes and culturing them in medium that is free of leukemia inhibitory factor (LIF), an inhibitor of differentiation, to generate patches of cardiomyocytes that exhibit spontaneous contractile activity (see U.S. Pat. No. 5,733,727, col. 12, lines 63-67). Field also described a useful method for purifying cells that have differentiated into a specific cell type from other types of cells present in the culture: the parental ES cells are cotransfected with a PGK-HYG plasmid containing a Hygro^{r} DNA sequence that confers resistance to hygromycin, and a plasmid containing a MHC-Neo^{r} fusion gene—an α-cardiac myosin heavy chain (MHC) promoter operably linked to a Neo^{r} gene that confers resistance to neomycin. The PGK-HYG plasmid provides selection for transfected cells, while the MHC-Neo^{r} gene permits a second round of selection of the differentiated cells—incubation in the presence of G418 eliminates non-cardiomyocyte cells in which the MHC promoter is inactive (see col. 12, lines 63-67). The disclosure of U.S. Pat. No. 5,733,727 is incorporated herein by reference in its entirety. Schuldiner et al. described a systematic approach to analyzing the differentiation of ES-derived cells in response to different growth factors. They cultured human ES cells to generate embryoid bodies, dissociated the embryoid bodies, and cultured the cells as a monolayer in the presence of one of eight different growth factors. The differentiation induced by the growth factors was examined by monitoring changes in the cells' morphologies, and by RT-PCR (reverse transcription—polymerase chain reaction) analysis of the expression of a panel of 24 cell-specific genes in the parental ES cells, embryoid body cells, and the dissociated embryoid body cells cultured in the presence or the absence of one of the eight growth factors. Schuldiner et al. reported that each of the growth factors appeared to induce expression of different subset of the 24 marker genes that were analyzed; and that the growth factor-treated cultures were relatively homogenous, often containing only one or two cell types, whereas the dissociated embryoid cells cultured in the absence of a growth factor spontaneously differentiated into many different types of colonies. The growth factors appeared to act more by inhibiting than by inducing the differentiation of specific cell types, and none of the growth factors tested directed a completely uniform and singular differentiation of cells, and suggesting that direction of formation of specific cell types will require combinations of factors including those that inhibit undesired pathways and those that induce differentiation of specific cell types. (See Proc. Natl. Acad. Sci. USA (2000) 97(21): 11307-12). Paquin et al. described culturing murine P19 ES cells under conditions resulting in formation of aggregates of cells, some of which differentiated into beating cardiomyocytes (Proc. Nat. Acad. Sci. (2002) 99(14):9550-9555). Reubinoff et al. described manipulating the conditions in which human ES cells were cultured to induce their differentiation directly into neural precursors that could then be induced to differentiate into derivatives of the three neural lineages, neuronal cells, glial cells, and astrocytes (Nature Biotechnology (2001) 19:1134-1139). Kelly et al. have shown that changes in gene expression in ES cells in response to retinoic acid are highly reproducible (Mol. Reprod. Dev. (2000) 56(2):113-23), a result that implies that growth factor-directed differentiation of embryonic cells is dependably reproducible. The disclosures of the forementioned articles by Schuldiner et al., Paquin et al., Reubinoff et al., and Kelly et al. are incorporated herein by reference in their entirety.

(2) Identification and characterization of developmentally regulated genes that increase or decrease in expression during differentiation:

A second approach taken to study the differentiation of stem cells into specific cell types has been to expose the stem cells to conditions in which they become partially or fully differentiated, and to identify and characterize genes that are transcriptionally activated or repressed as the cells undergo differentiation. A number of research groups are pursuing this approach by making libraries of gene trap ES cells, and assaying to detect changes in the level of expression of the gene trap markers as the ES cells differentiate in vitro and in vivo. Methods for making gene trap cells and for detecting changes in the expression of the gene trap markers as the cells differentiate are reviewed in Durick et al. (Genome Res. (1999) 9:1019-25), the contents of which are incorporated herein by reference in its entirety. Vectors and methods useful for making gene trap ES cells and for detecting changes in the expression of the gene trap markers as the cells differentiate are also described in U.S. Pat. No. 5,922,601 (Baetscher et al.) and in U.S. Pat. No. 6,248,934 (Tessier-Lavigne), the contents of which are also incorporated herein by reference in their entirety. Murine ES cells are the stem cells that are widely used in large-scale gene trap studies. Methods for producing murine ES cells, for genetically modifying them, for inducing their differentiation in vitro, and for using them to generate chimeric or nuclear-transfer cloned embryos and cloned mice are developed and known in the art. To facilitate the identification of genes and the characterization of their physiological activities, large libraries of murine gene trap ES cells having gene trap DNA markers randomly inserted in their genomes have been prepared, and efficient methods have been developed to screen these and detect changes in the level of expression of the gene trap markers as the ES cells differentiate in vitro or in vivo.

For example, a library of gene trap murine ES cells called OmniBank® that is available from Lexicon Genetics, Inc. (The Woodlands, Tex.) is reported to contain more than 200,000 frozen mouse embryonic stem cell (ES) clones, each identified in a relational database by DNA sequence from the knocked-out gene, and is estimated to contain gene knockout clones for more than 50 percent of the genes in the mammalian genome. The German Gene Trap Consortium (GGTC) has been established in Germany to provide a reference library of gene trap sequence tags (GTST) in mouse ES stem cells. See Wiles et al. (Nature Genetics, 2000, 24(1)), incorporated by reference in its entirety. The public aims of the GGTG are the generation of a library of 20,000 gene trap ES cell integrations, cloning and sequencing of the tagged genes, generation of animal models, and analysis of mutant phenotypes resulting from gene knockouts. Sequence information on the GTST library is accessible at the Internet site of the GGTC, and the mutant ES cells are available upon request. Publicly available libraries murine gene trap ES cell lines are also being developed at the Gene Trap Laboratory of The Centre for Modeling Human Disease in Toronto, Canada, and by the Bay Genomics project, a collaboration of researchers at the University of California-Berkeley, the University of California-San Francisco, and Stanford University, with ties to the National Heart, Lung, and Blood Institute's (NHLBI) genomics program. In 2001, it was estimated that more than 8000 gene trap ES cell lines were freely available (Stanford, Nat. Rev. Genet., 2001, 2(10): 756-68).

The screening and analysis of gene trapped stem cells is generally carried out as follows. First, a “library” of gene trapped stem cells is prepared by insertion of gene trap marker DNA constructs into multiple genes in the cells' genomic DNA. Typically, any given cell in the population of cells making up the library will have, at most, one construct inserted into its genome. The gene trap markers are typically inserted randomly, so that only a subset of the genetically altered stem cells have gene trap markers inserted at locations and with orientation such that they are under transcriptional control of an endogenous promoters of a genes. Selection of stem cells containing a stable transfection of a gene trap marker can be facilitated by using a gene trap marker that includes DNA encoding a selectable marker, e.g., a protein that confers resistance to an antibiotic, and by culturing the stem cells in medium containing an agent that selects against the cells in which the marker is not expressed, as described by Baker et al. (Dev. Biol. (1997) 185(2):201-214). The colonies of cells that survive selection are called gene trapped ES clones.

Stem cell clones are then subjected to additional analysis; e.g., by screening in vivo by making chimeric or cloned embryos and monitoring the expression of the gene trapped markers in embryonic and fetal tissues during development and differentiation of the cell types of interest, to see if the expression of the gene trapped markers in vivo correlates with the pattern observed in vitro (Stanford et al., Blood (1998) 12:4622-2631; also Bonaldo et al., Exp. Cell Res. (1998) 244(1):125-136); by isolating and determining the nucleotide sequences of the marked genes, and by assaying to identify the physiological functions of the proteins encoded by the marked genes. Secondary analyses of selected gene trapped stem cells can be performed using known methods; e.g., the nucleotide sequences of the marked genes can be determined by the method of 5′-rapid amplification of cDNA ends (5′-RACE) (Voss et al., Dev. Dyn. (1998) 212(2):171-180), or by cloning marked RNA transcripts and sequencing the resulting cloned DNA molecules (Holzchu et al., Transgenic Res. (1997) 6(1):97-106). Analysis of expression of gene trap markers in vivo can be performed using in situ hybridization (see Baker et al., 1997, supra). Using known methods, hybridization probes that hybridize specifically to the marker nucleotide sequences, or to transcripts encoded by the locus marked by the gene trap marker, can be labeled to produce a fluorescent signal and used to perform fluorescent in situ hybridization (FISH) to detect expression of marked genes in cells in vivo (e.g., in tissue sections), as well as in cells cultured in vitro. Using known methods, marked cells can also be detected using labeled antibodies that specifically bind to marker proteins expressed by gene trap DNA constructs, or to marker proteins expressed by the genes that have been marked by gene trap constructs. Insertion of a gene trap marker DNA into a gene frequently disrupts the marked locus and prevents it from being effectively expressed. Chimeric mice, their offspring, or cloned mice generated from gene trap ES cells that are heterozygous for a locus that is silenced by marker insertion can be interbred to produce mice that are homozygous for the marked locus, and analysis of the effects of the null mutation on the development of such mice can provide information about the physiological function of the marked locus (Forrester et al., Proc. Nat. Acad. Sci. (1996) 93:1677-1682; also Voss et al., 1998, supra).

The above-described approaches for studying the factors that regulate and control stem cell differentiation provide information about conditions under which stem cells differentiate, and focus on the roles of specific genes that are transcriptionally activated during development; but they do not provide a systematic method for identifying the sets of sequential transcriptional regulatory events that occur over time when stem cells differentiate of into each of the different cell types of a mammal. More importantly for generating differentiated cells and tissues for transplant therapy, the above-described approaches do not provide information relating to the differentiation of stem cells ex vivo, such as in tissue culture.

### BRIEF DESCRIPTION OF THE FIGURES

FIG. 1 schematically depicts a developmental tree representing differentiation of a totipotent stem cell into multiple different cell types. Each “branch” of the developmental tree that runs from the stem cell to a differentiated cell type is made up of a series of linked nodes that represents the temporal sequence of transcriptional regulatory events that occur in the cell lineage generated when the stem cell differentiates into that cell type.

FIG. 2 graphically represents the temporal patterns of the activation and repression of transcription of genes that are conditionally expressed in totipotent stem cells (e.g., ES cells) that are exposed to conditions that induce differentiation into one or more cell types.

FIGS. 3A and 3B schematically represent data obtained by disclosed methods for “mapping” or “walking” a branch of the developmental tree.

FIG. 3A depicts nodes of the developmental tree that correspond to expression of genes in a differentiating cell. The nodes labeled “A” and “C” correspond to genes that are expressed early and late, respectively, in cells that are differentiating into a cell type of interest. The node labeled “E” corresponds to a gene that is expressed late in cells that are differentiating into a different cell type. The dotted line extending from node A to node C represents the temporal and spatial region of interest, and corresponds to the expression of unidentified genes that are transcriptionally activated in the lineage of cells differentiating into the cell type of interest.

FIG. 3B depicts the portion of the developmental tree shown in FIG. 3A, with the addition of the node labeled “B” corresponding to a gene identified by the invention that is transcriptionally activated after gene A and before gene C in cells that are differentiating into the cell type of interest.

FIGS. 4A and 4B schematically also represent data obtained by disclosed methods for mapping a branch of the developmental tree.

FIG. 4A depicts a branch the developmental tree that correspond to the differentiation of a stem cell into a particular cell type of interest. The node labeled “Q” corresponds to a gene that is expressed late in cells that are differentiating into the cell type of interest, and the dotted line extending from the stem cell to node Q represents the temporal and spatial region of interest, and corresponds to the expression of unidentified genes that are transcriptionally activated in the lineage of cells differentiating into the cell type of interest.

FIG. 4B depicts a set of nodes of the developmental tree identified by using the invention to detect multiple clones that co-express gene Q and various different genes in cells differentiating into the cell type of interest. By monitoring the timing of expression of these genes, the nodes corresponding to their expression can mapped to the branch of the developmental tree corresponding to differentiation of the cell type of interest, as shown at the bottom of FIG. 4B.

### DESCRIPTION OF THE INVENTION

### Overview of the Invention

Chimeric or completely transgenic mice can be generated from cloned single cell colony-derived gene trap ES cells using known methods, and the mice can be screened to observe the distribution of cells in which the marker genes are expressed, as described above. Using this technique, gene trap cells of such mice showing cell type-specific, tissue-specific, and/or developmental stage-specific patterns of expression (e.g., expression of the gene trap marker gene only in kidney) can be identified, and the nucleotide sequences of the genes into which the markers were inserted can be determined, e.g., by 5′ RACE or similarly effective molecular techniques known in the art. Such techniques have been shown to be useful in identifying new genes, determining their nucleotide sequence, and characterizing their role(s) in development. The present invention describes screening methods that can use such gene trapped stem cells in measuring temporal and spatial patterns of gene expression in specific types of differentiating cells in vivo and in vitro, and it describes methods in which lines of such gene trapped stem cells are used in stem cell research and in developing cell-based therapies.

New screening methods that make use of gene trapped cell lines and provide means for efficiently identifying combinations of biological, biochemical, and physical agents or conditions that influence the differentiation of totipotent or pluripotent stem cells are described in co-owned and co-pending U.S. Application No. PCT/US02/26945, filed Aug. 26, 2002, entitled “Screening Assays for Identifying Differentiation-Inducing Agents and Production of Differentiated Cells for Cell Therapy,” the contents of which are incorporated herein by reference in their entirety.

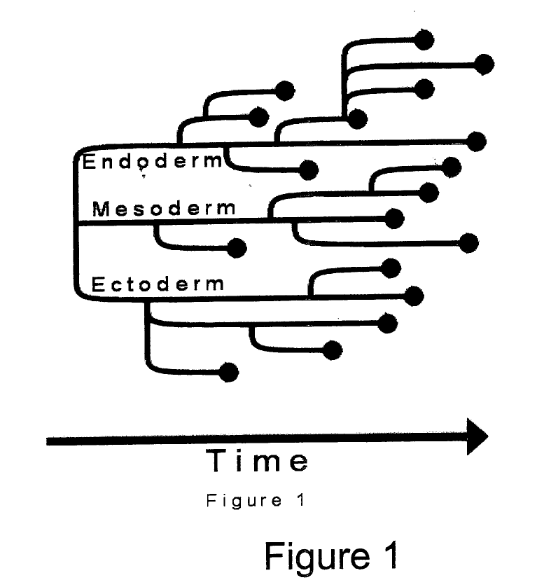

The development of a multicellular organism from an embryo comprising a few totipotent or nearly totipotent stem cells proceeds by a temporally regulated, genetically determined process in which the stem cells divide and give rise to progressively committed cell lineages that differentiate into all of the different cell types of an animal. This process can be regarded abstractly as giving rise to a “developmental tree” formed by multiple branching pathways made up of linked “nodes” that correspond to the sequential transcriptional regulatory events that direct the differentiation of totipotent stem cells into pluripotential stem cells, and the subsequent differentiation of these into the many different fully differentiated cell types of a developed animal. A schematic depiction of a developmental tree is shown in FIG. 1. The tree's branching pathways of linked nodes that correspond to the multiple series of timed genetic or epigenetic regulatory events (e.g., activation or repression of specific genes) that occur in cell lineages that are generated when the stem cell divides and differentiates to form the different types of partially and fully differentiated cells in an animal's body.

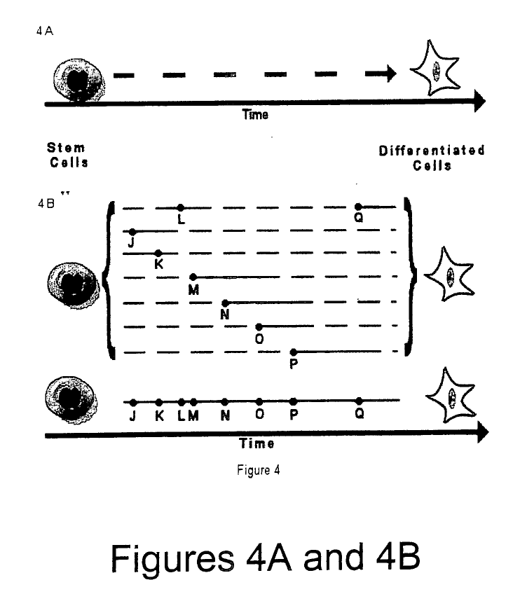

As described in detail below, one object of the present invention is to provide methods for identifying the genes that are sequentially expressed in cells of a specific lineage that are differentiating to give rise to a particular cell type of interest ex vivo, such as a pluripotential stem cell (e.g., a multipotential precursor cell), a particular type of precursor cell committed to form one or a few specific cell types, or a particular fully or terminally differentiated cell type. The methods of the present invention permit the generation of temporal maps similar to that shown in FIG. 4B that depict the temporal patterns of the expression of specific genes that are conditionally expressed during the development and differentiation of each of the different cell types of an animal ex vivo.

### Embodiments of the Method of the Present Invention Comprise

(a) obtaining the means for detecting the expression of genes that are transcriptionally activated and/or repressed in cells in the course of differentiation of stem cells ex vivo or in vivo into different types of partially and fully differentiated cell types of the body (this step constitutes “primary screening”); and then

(b) monitoring the expression of these genes in specific types of differentiating cells ex vivo or in vivo during embryological and/or fetal development to detect the activation and/or repression of transcription of the genes that occurs sequentially over time when stem cells differentiate into each of the different cell types of an animal, and correlating the relative timing of these genetic regulatory events with the generation of the different types of partially differentiated, pluripotent progenitor cells and fully differentiated cell types that arise during development (steps of “secondary screening”).

An object of these embodiments of the invention is to identify, for any partially or fully differentiated cell type of interest, a specific set of conditionally expressed genes, the members of which are transcriptionally activated in a sequential manner in cells produced by differentiation of stem cells in vivo into the partially or fully differentiated cell type.

Another object is to determine the temporal pattern of the transcriptional activation and/or repression of the specific set of genes that occurs in cells that are differentiating in vivo into a partially or fully differentiated cell type of interest.

A third object of the invention is to analyze and characterize the cell types, e.g., histologically and/or biochemically, in the lineage formed by differentiation of stem cells in vivo into a cell type of interest, and to correlate the appearance, biochemical markers, and physical location of these partially and/or fully differentiated cell types with the temporal pattern of the expression of the set of genes that is conditionally expressed in these cells during their differentiation. The lineage-specific information obtained by the above-described embodiments of the invention can be thought of as genetic, temporal, and histological correlates to a single “branch” of the developmental tree shown in FIG. I that leads from a stem cell to a particular differentiated cell type of interest.

A fourth object is to identify nodes of a single “branch” of the developmental tree that leads from a stem cell to a particular differentiated cell type of interest, to allow screening to identify conditions or factors that induce stem cells to differentiate in vitro into the cell type of interest.

A fifth object of the invention is to use developmental stage-specific markers to isolate cells that are differentiated to a stage of differentiation of interest, for the purpose of screening to identify conditions or factors that induce cells to differentiate from one stage of differentiation to another, and for identifying differentiation antigens that facilitate purification of cells that are differentiated to a stage of differentiation of interest.

### Primary Screening to Identify Conditionally Expressed Genes

Primary screening steps of the present invention identify some or all of the genes of an animal that are conditionally expressed during the differentiation of stem cells in vivo and ex vivo into the partially and fully differentiated cells of the animal's body. The primary screening methods of the present invention can also identify conditionally expressed genes of animal cells that show other unique and useful patterns of gene expression; e.g., conditional expression related to control of proliferation or cell survival.

The primary screening phase of the present invention is efficiently performed by making libraries of gene trapped stem cells, inducing these to differentiate ex vivo and in vivo, and monitoring to identify the cells containing trapped genes that are conditionally expressed in the differentiating cells. DNA sequences of conditionally expressed genes can then be isolated and their nucleotide sequences determined.

Alternatively, the primary screening steps of the present invention can also be performed by using presently available techniques of molecular biology. For example, using DNA microarray analysis or conventional hybridization assay methods and hybridization probes complementary to coding sequences identified by analysis of genomic DNA sequences and expressed sequence tags, mRNAs isolated from differentiating cells at various stages of differentiation in vivo or ex vivo can be analyzed quantitatively to identify genes that are conditionally expressed in differentiating cells.

### Secondary Screening

Secondary screening methods of the invention are used to monitor the transcriptional activities of conditionally expressed genes in specific types of differentiating cells in vitro and in vivo. The invention can be used to monitor the expression of conditionally expressed genes in vivo during the development of an embryonic, fetal, and adult mammal, to identify, for each differentiated cell type of a mammal, a set of conditionally expressed genes that are transcriptionally activated at different times during the differentiation of stem cells into the differentiated cell type. The invention further allows one to correlate the relative timing of these genetic events with the generation of the different types of partially and fully differentiated cell types that arise during development of a mammal. The secondary screening methods comprise, for a particular partially or fully differentiated cell type of a mammal, inducing a population of stem cells to differentiate into a particular cell type; and assaying to detect conditionally expressed genes identified in the primary screening step that are transcriptionally activated in cells that are differentiating into the particular cell type of interest. The secondary screening methods are efficiently performed using gene trapped stem cells containing trapped genes that are shown in primary screening to be conditionally expressed in differentiating cells. The gene trapped stem cells are induced to differentiate in vivo and/or in vitro, and histological and molecular biological methods are used to:

(a) identify the gene trapped stem cells having genes that are transcriptionally activated or repressed at different times in cells of a lineage formed by differentiation of the stem cells into the particular cell type of interest,

(b) characterize the conditionally expressed genes in terms of the timing of their transcriptional activation and/or repression, and

(c) describe and characterize the different type of cells and tissues in which the genes are expressed during their development and differentiation into the particular cell type of interest.

### Applications of the Invention

The primary and secondary screening methods of the invention allow one to identify multiple developmentally regulated genes that are transcriptionally activated at different times during differentiation of a stem cell into a particular cell type of interest. The cell type of interest may be either a partially differentiated cell type, or a fully or terminally differentiated cell type. The information obtained with these screening methods can be used to identify conditions or factors that induce stem cells to differentiate in vitro into such partially or fully differentiated cell types of interest. It also permits one to isolate populations of cells that are uniformly differentiated into a particular cell type of interest. Such purified preparations of differentiated cells are valuable for basic research, for drug discovery and testing, and for toxicological studies. They are particularly useful in studying cell-cell interactions that play a role in inducing and regulating the differentiation of cells into particular cell types, and for producing antibodies that specifically bind differentiation antigens that are specifically produced by partially or fully differentiated cell types of interest. Such antibodies are useful for isolating purified preparations of partially or fully differentiated cell types of interest that are free of genetic modification and are useful for cell therapy in humans.

### Terms used in the Application

As used herein, a “stem cell” is a cell that has the ability to proliferate in culture, producing some daughter cells that remain relatively undifferentiated, and other daughter cells that give rise to cells of one or more specialized cell types; and “differentiation” refers to a progressive, transforming process whereby a cell acquires the biochemical and morphological properties necessary to perform its specialized functions. Stem cells therefore reside immediately antecedent to the branch points of the developmental tree.

As used herein, an “embryonic stem cell” (ES cell) is a cell line with the characteristics of the murine embryonic stem cells isolated from morulae or blastocyst inner cell masses (as reported by Martin, G., Proc. Natl. Acad. Sci. USA (1981) 78:7634-7638; and Evans, M. and Kaufman, M., Nature (1981) 292: 154-156); i.e., ES cells are capable of proliferating indefinitely and can differentiate into all of the specialized cell types of an organism, including the three embryonic germ layers, all somatic cell lineages, and the germ line.

As used herein, an “embryonic stem-like cell” (ES-like cell) is a cell of a cell line isolated from an animal inner cell mass or epiblast that has a flattened morphology, prominent nucleoli, is immortal, and is capable of differentiating into all somatic cell lineages, but when transferred into another blastocyst typically does not contribute to the germ line. An example is the primate “ES cell” reported by Thomson et al. (Proc. Natl. Acad. Sci. USA. (1995) 92:7844-7848).

As used herein, “inner cell mass-derived cells” (ICM-derived cells) are cells directly derived from isolated ICMs or morulae without passaging them to establish a continuous ES or ES-like cell line. Methods for making and using ICM-derived cells are described in co-owned U.S. Pat. No. 6,235,970, the contents of which are incorporated herein in their entirety.

As used herein, an “embryonic germ cell” (EG cell) is a cell of a line of cells obtained by culturing primordial germ cells in conditions that cause them to proliferate and attain a state of differentiation similar, though not identical to embryonic stem cells. Examples are the murine EG cells reported by Matsui et al, 1992, Cell 70: 841-847 and Resnick et al, Nature. 359: 550-551. EG cells can differentiate into embryoid bodies in vitro and form teratocarcinomas in vivo (Labosky et al., Development (1994) 120:3197-3204). Immunohistochemical analysis demonstrates that embryoid bodies produced by EG cells contain differentiated cells that are derivatives of all three embryonic germ layers (Shainblott et al., Proc. Nat. Acad. Sci. U.S.A. (1998) 95:13726-13731).

As used herein, a “totipotent” cell is a stem cell with the “total power” to differentiate into any cell type in the body, including the germ line following exposure to stimuli like that normally occurring in development. An example of such a cell is an ES cell, an EG cell, an ICM-derived cell, or a cultured cell from the epiblast of a late-stage blastocyst.

As used herein, a “nearly totipotent cell” is a stem cell with the power to differentiate into most or nearly all cell types in the body following exposure to stimuli like that normally occurring in development. An example of such a cell is an ES-like cell.

As used herein, a “pluripotent cell” is a stem cell that is capable of differentiating into multiple somatic cell types, but not into most or all cell types. This would include by way of example, but not limited to, mesenchymal stem cells that can differentiate into bone, cartilage and muscle; hemotopoietic stem cells that can differentiate into blood, endothelium, and myocardium; neuronal stem cells that can differentiate into neurons and glia; and so on.

As used herein, “ex vivo” cell culture refers to culturing cells outside of the body. Ex vivo cell culture includes cell culture in vitro, e.g., in suspension, or in single- or multi-well plates. Ex vivo culture also includes co-culturing cells with two or more different cell types, and culturing in or on 2- or 3-dimensional supports or matrices, including methods for culturing cells alone or with other cell types to form artificial tissues.

As used herein, the term “gene” refers to the nucleotide sequences at a genetic locus that encode and regulate expression of a functional mRNA molecule or a polypeptide; i.e., as used herein, a gene includes the nucleotide sequences that make up the coding sequence (exons and introns), the promoter, enhancers, and other DNA elements that regulate transcription, including as elements conferring cell type-specific and differentiation stage-specific expression, hormone responsive elements, repressor elements, etc., and nucleotide sequences that encode signals that regulate splicing and translation of the mRNA, such as a cleavage signal, a polyadenylation signal, or an internal ribosome entry site (IRES).

As used herein in describing a nucleic acid sequence such as a promoter or coding sequence, “endogenous” refers a natural sequence in its native location in the genomic DNA of the cell; whereas “exogenous” refers to a DNA sequence that is inserted into the genomic DNA of a cell; e.g., as part of a recombinant DNA construct introduced by human industry. An exogenous DNA construct that is introduced into the genomic DNA of a cell can contain nucleotide sequences that were originally isolated from the same cell or species, or from a different cell or species.

As used herein, a “marker” is a detectable characteristic, feature, activity, or molecule of a cell that is indicative of a particular cellular state. A marker can be a biochemical entity that changes state in a detectable manner when the cell enters or leaves a particular state. The term “marker” as used herein can refer to a marker gene, or to a marker RNA or protein encoded by such a gene. For example, a marker can be a DNA sequence encoding a detectable product, e.g., a specific mRNA or antigenic protein, a fluorescent protein, a chromogenic enzyme, or a protein conferring antibiotic resistance. Any DNA encoding a product that allows the detection of cells in which the construct is expressed can be used as a detectable marker for the present invention. For example, a DNA that encodes a recombinase enzyme is referred to herein as encoding a detectable product if the recombinase produced by expression of the DNA in a cell catalyzes a recombination reaction (e.g., excision of an inhibitory DNA sequence) that activates expression of a different gene that encodes a product that produces a detectable signal.

### Stem Cells

The stem cells made by and used for the methods of the present invention may be any appropriate totipotent, nearly totipotent, or pluripotent stem cells. Such cells include inner cell mass (ICM) cells, embryonic stem (ES) cells, embryonic germ (EG) cells, embryos consisting of one or more cells, embryoid body (embryoid) cells, morula-derived cells, as well as multipotent partially differentiated embryonic stem cells taken from later in the embryonic development process, and also adult stem cells, including but not limited to nestin positive neural stem cells, mesenchymal stem cells, hematopoietic stem cells, pancreatic stem cells, marrow stromal stem cells, endothelial progenitor cells (EPCs), bone marrow stem cells, epidermal stem cells, hepatic stem cells and other lineage committed adult progenitor cells.

Totipotent, nearly totipotent, or pluripotent stem cells, and cells therefrom, for use in the present invention can be obtained from any sources of such cells. One means for producing totipotent, nearly totipotent, or pluripotent stem cells, and cells therefrom, for use in the present invention is via nuclear transfer into a suitable recipient cell as described, for example, in co-owned U.S. Pat. No. 5,45,577, and U.S. Pat. No. 6,215,041, the disclosures of which are incorporated herein by reference in their entirety. Nuclear transfer using an adult differentiated cell as a nucleus donor facilitates the recovery of transfected and genetically modified stem cells as starting materials for the present invention, since adult cells are often more readily transfected than embryonic cells. Other aspects of cloning by nuclear transfer leading to production of totipotent, nearly totipotent, or pluripotent stem cells, are also described in the co-owned and co-pending U.S. Patent Applications that are listed above in the section of the application describing the background of the invention, and are also incorporated herein by reference.

The methods of the invention may be performed with totipotent, nearly totipotent, or pluripotent stem cells, and cells therefrom, of any animal species, including but not limited to human and non-human primate cells, ungulate, canine, feline, lagomorph, rodent, avian, and fish cells. Primate cells with which the invention may be performed include but are not limited to cells of humans, chimpanzees, baboons, cynomolgus monkeys, and any other New or Old World monkeys. Ungulate cells with which the invention may be performed include but are not limited to cells of bovines, porcines, ovines, caprines, equines, buffalo and bison. Rodent cells with which the invention may be performed include but are not limited to mouse, rat, guinea pig, hamster and gerbil cells. Rabbits are an example of a lagomorph species with which the invention may be performed. Chickens (Gallus gallus) are an example of an avian species with which the invention may be performed.

For example, the methods of the invention may be performed with murine ES cells lines, or with primate ES or EG cell lines. An example of a parthenogenetically derived primate stem cell line with which the methods of the invention may be performed is the totipotent non-human primate stem cell line Cyno-1, which was isolated from the inner cell mass of parthenogenetic Cynomologous monkey embryos and is capable of differentiating into all the cell types of the body. Cibelli et al. (Science (2002) 295:819).

It is expected that the earliest and most important applications of the present invention will be performed with mammalian cells. Accordingly, although the methods of the present invention are suitable for mammalian and non-mammalian species alike, the present application will henceforth refer to the method as being performed with or applied to cells of a mammal.

### Marker DNA Constructs

Marker DNA constructs useful for the present invention include genes encoding products that are detectable by any means, i.e., those that are detectable by the naked eye or after microscopic, photographic or radiographic analysis, or after contacting said exposed cells with a reagent selected from the group consisting of chromogenic substrates, dyes, sugars, antibodies, ligands, primers, etc. Suitable marker DNA constructs include DNA sequences encoding mRNAs that are detectable with labeled DNA or RNA hybridization probes, and DNA sequences encoding proteins that are detectable with labeled antibodies. Many detectable markers that are suitable for use in the present invention are known and are presently available; suitable reporter genes may encode polypeptides including but not limited to green fluorescent protein (GFP), enhanced green fluorescent protein (EGFP), luciferase, chloramphenicol acetyltransferase, β-glucuronidase, β-galactosidase, alkaline phosphatase, guanine xanthine phosphoribosyltransferase or β-lactamase. See, e.g., U.S. Pat. No. 5,928,88, herein incorporated by reference. GFP is a widely used fluorescent marker that is available in variants that emit light of blue, cyan, green, and yellow wavelengths. The use of a marker gene encoding a fluorescent protein such as GFP permits detection of expression of the marker gene without injuring the cells. E.coli lacZ gene (encoding a β-galactosidase) can be used as either a chromogenic or a fluorogenic marker, depending on the substrate that is provided. Fluorogenic substrates suitable for use in the present invention include but are not limited to fluorescein di-β-D-galactopyranoside, resorufin β-D-galactopyranoside, DDAO galactoside, methylumbelliferyl galactoside or its di-fluorinated analog, carboxyumbelliferyl galactoside, fluorescent glycolipids, Amplex Red Galactose, PFB Aminofluorescein, chloromethyl and lipophilic derivatives of DiFMUG, 4-methylumbelliferyl β-D-glucuronide, fluorescein di β-D-glucuronide, 5-(pentafluorobenzoylamino)fluorescein di β-D-glucuronide, DDAO β-D-glucuronide, etc. Those skilled in the art are familiar with many reagents for detecting glycosidase activity.

Marker DNA constructs suitable for the present invention also include DNA sequences encoding a selectable marker, e.g., a protein that confers resistance to a selecting agent that selects against non-resistant cells. Methods using DNA constructs encoding selectable markers to select cells are well known in the art; examples of suitable selectable markers include DNA sequences encoding neomycin phosphotransferase (Neo^{r}) and hygromycin phosphotransferase (Hygro^{r}) that confer resistance to the antibiotics neomycin and hygromycin, respectively.

A useful pair of marker DNA constructs that can be used together to select and isolate a population of cells that are differentiated into a specific cell type is described by Field (U.S. Pat. No. 5,733,727, incorporated herein), as discussed above. One marker DNA construct contains a PGK-Hygro^{r} cassette—a broad specificity phosphoglycerate kinase promoter operably linked to a DNA sequence encoding Hygro^{r} for general selection of transfected cells. The other marker DNA construct contains a MHC-Neo^{r} cassette—a cardiomyocyte-specific α-cardiac myosin heavy chain promoter operably linked to a DNA sequence encoding Neo^{r} for selection of transfected cardiomyocytes.

### Making and Modifying Gene Trapped Stem Cells

An assortment of different vectors and methods useful for making gene trapped stem cells and for detecting changes in the expression of the gene trap markers in cells derived from the making gene trapped stem cells have been described (see Background). Genetically modified stem cells useful for present invention can be produced using known methods. For example, a library of murine gene trap ES cells useful in the primary and secondary screening methods of the present invention can be made by randomly inserting marker DNA constructs into the genomic DNA of murine ES cells, inducing the ES cells to differentiate, and selecting ES cells giving rise to differentiating cells in which the marker constructs are expressed, as discussed above.

A promoterless marker DNA construct that is useful for making gene trap cells to detect differentiation-specific genes encodes mRNA that functions as a discrete cistron and is inserted into a gene at a site downstream of the endogenous promoter and comprises an internal ribosome entry site (IRES) or is inserted in-frame so that it is transcriptionally regulated by the endogenous promoter and its associated regulatory sequences (such constructs have also been referred to as “promoter traps”). Promoterless DNA marker constructs comprising an IRES for efficient expression following insertion into a gene are described, for example, in Mountford et al. (Proc. Nat. Acad. Sci. (1994) 91:4303-4307), the contents of which are incorporated herein by reference.

A marker DNA construct comprising two or more separately transcribed expression cassettes can be also used in the present invention. For example, a DNA construct could contain a promoterless cassette comprising an IRES and a DNA sequence encoding GFP for marking (trapping) a developmentally regulated gene, and another cassette comprising a constitutive or broad specificity promoter such as a PGK or cytomegalovirus (CMV) promoter that is operably linked to a DNA sequence encoding a protein conferring antibiotic resistance for selection, or a fluorescent protein (e.g., blue fluorescent protein) different from that encoded by the promoterless cassette, for visually distinguishing the tagged cells from untagged cells.

Another example of a marker DNA construct that is useful in the present invention is a promoterless construct that includes sequences that mediate fusion of the marker mRNA to an mRNA encoded by one or more exons of the gene in which the marker DNA is inserted; e.g., a promoterless construct consisting of a splice acceptor site followed by a reporter gene and a poly-adenylation sequence (reviewed in Durick et al., Genome Res., 1999, 9:1019-25). Such a marker construct can be inserted into an intron so that it is transcriptionally regulated by the endogenous promoter, and its transcription produces a fusion mRNA formed by splicing of a splice donor sequence at the end of an endogenous exon with the splice acceptor sequence of the marker mRNA (see Durick et al.,1999).

Insertion of the above-described gene trap marker DNA constructs into the trapped gene usually prevents it from being expressed effectively, thereby “knocking out” the trapped gene; however, there are reports that such randomly inserted gene trap marker constructs can be co-expressed with the gene in which they are inserted without inhibiting the expression of the tagged gene (McClive et al., Dev. Dyn., 1998, 212(2):267-276; Voss et al., Dev. Dyn., 1998, 212(2):258-266; Voss et al., Dev. Dyn., 1998, 212(2):171-180).

While knocking out the expression of one allele of a genetic locus of a stem cell, referred to herein as a heterozygous knockout, frequently has no apparent effect on the differentiation of the stem cell, in some cases it can delay or change the course of differentiation of the stem cell and disrupt normal embryonic or fetal development. As described below, gene trapped stem cells that are produced for the present invention in which a DNA marker construct is inserted to effect a heterozygous knockout can also be useful intermediates for producing stem cells in which both copies of the allele in the cell are knocked out, referred to herein as a homozygous knockout. For example, DNA of the gene in which the DNA marker construct is inserted can be cloned and used to disrupt the second copy of the allele in the stem cell line by homologous recombination. Stem cells with homozygous knockouts of genes can be used to produce differentiated cells or generate cloned animals that are of great value as experimental model systems for studying and testing drugs and other treatments for diseases associated with the mutation or loss of the genes.

Generation of gene trapped stem cells by randomly inserting promoterless marker DNA constructs and screening for cells containing differentiation-specific markers is an approach that is useful for large-scale, high-throughput screening to detect differentiation-specific genes. However, gene trapped stem cells useful for the present invention can also be made using homologous recombination to precisely insert a marker DNA construct into a selected site in a gene that is conditionally expressed in a differentiating cell. The DNA construct to be inserted can be designed to disrupt and inhibit expression of the endogenous gene, producing a heterozygous knockout; or it can be designed to be transcribed as a separate cistron without disrupting expression of the endogenous coding sequence, e.g., by including an IRES in the marker DNA construct and inserting it downstream of the endogenous stop codon (see Mountford et al., 1994).

Gene trap marker DNA constructs encoding a single detectable marker such as GFP or lacZ, or a fusion of one of these with a selectable marker such as Neo^{r} or Hygro^{r} (encoding hygromycin resistance) are useful for the methods of the present invention, as described below. Bicistronic or multicistronic marker DNA constructs encoding a single mRNA that has two or more separately translated open reading frames, each of which has the regulatory signals (e.g. an IRES sequence) necessary for its expression, are also useful for the present invention. Expression vectors containing IRES elements that can be used to make such multi-cistronic marker DNA constructs are commercially available; for example, the pMACS 4-IRES (Miltenyi Biotec, Auburn, Calif.) and pIRESbleo3 (BD Biosciences, Palo Alto, Calif.) expression vectors both contain the IRES element of the encephalomyocarditis virus. For example, a bicistronic marker DNA construct with cistrons encoding GFP and Hygro^{r} allows the identification of clones in which the marker is expressed by detection of GFP fluorescence, and purification of the marked clones by using hygromycin to selectively eliminate the unmarked cells. Purification using a selectable marker is useful, because it allows isolation of the marked cells without having to trypsinize them to remove them from their dish, a step that may alter the surface of the cells with undesirable consequences, as discussed below.

A gene trap marker DNA construct useful for the present invention can also include DNA sequences comprising a promoter operably linked to a reporter gene, but lacking a polyadenylation sequence. Expression of such a construct in a cell is evidence that the construct is inserted into a coding sequence that has a functional polyadenylation sequence. Such marker DNA constructs are therefore useful for identifying and isolating gene trap cells having marker DNA constructs inserted into genes (see Zambrowicz et al., U.S. Pat. No. 6,080,576, the contents of which are incorporated herein by reference in entirety).

Gene trap marker DNA constructs useful for the present invention can also include two or more coding sequences that are under control of different promoters. For example, a marker DNA construct can comprise a promoterless cassette encoding a detectable marker such as GFP, and another cassette comprising an exogenous constitutive promoter operably linked to a nucleotide sequence encoding a different detectable marker, such as cyan fluorescent protein (CFP), an antibiotic resistance gene or, such as Neo^{r}, or a bicistronic cassette encoding both CFP and Neo^{r} for distinguishing gene trap cells from other cells, and for tracking them in vivo. Alternatively, the second cassette can comprise an exogenous cell type-specific or developmental stage-specific promoter that directs expression in a cell-type of interest; e.g., for screening to identify genes that are also conditionally expressed in the same cell type, as described herein. Exogenous promoters useful in the present invention can be of the same species as the stem cell, or of a different species.

Embodiments of the present invention also include gene trapped stem cells that are genetically modified by insertion of marker DNA constructs into multiple sites in the same stem cell. For example, marker DNA constructs encoding different detectable markers (e.g., color variants of GFP) can be inserted by homologous recombination into selected sites in each of a set of genes that are shown by the methods of the present invention to be expressed in cells that are differentiating into the same cell type. The marker DNA constructs can be inserted at sites selected to disrupt expression of the targeted genes, or at sites selected to avoid inhibiting expression of the genes, as discussed above.

The present invention also includes embodiments gene trapped stem cells are genetically modified by insertion of a DNA expression cassette encoding a recombinase into their genomic DNA. Methods for using DNA sequences encoding recombinase and recombination sites recognized by the recombinase to activate or inhibit expression of other DNA constructs are well known; examples of recombinase systems that are suitable in the present invention include the well-characterized Cre-lox and the Flp-FRT systems (see Ryding et al., J. Endocrinology, 2001, 171:1-14; and Dymecki et al., Proc. Nat. Acad. Sci., U.S.A., 1996, 93:6191-6196, the contents of which are incorporated herein by reference in their entirety).

Embodiments of the present invention can be used to permanently mark differentiating cells in which a gene of interest is transiently expressed. An example of such embodiments comprises inserting a DNA construct comprising a promoterless DNA expression cassette encoding a recombinase into a site in a gene of a stem cell that is transiently expressed in cells differentiating into a particular cell type, so that expression of the DNA encoding recombinase is under control of the endogenous promoter of the gene in which it is inserted. Another expression cassette is also inserted into the genomic DNA of the same stem cell, either in the same or in a different DNA construct, that comprises (i) a consititutively activated promoter, (ii) a marker DNA sequence encoding a detectable product, and (iii) a nucleotide sequence flanked by two recombination sites that is inserted between the constitutive promoter and the marker DNA sequence, and inhibits expression of the marker DNA sequence. In this embodiment, transiently expression of the gene in which the recombinase cassette is inserted results in expression of the DNA sequence encoding recombinase, and excision by the recombinase of the inhibitory nucleotide sequence from between the promoter the sequence encoding the detectable product. The constitutive promoter then directs transcription of the sequence encoding the detectable product, so that the cell continues to produce a detectable signal even after the transiently activated promoter becomes inactive. Embodiments of the invention using this method or its variants are useful for following the fate of a differentiating cell in vivo or ex vivo; e.g., for identifying the set of partially differentiated cell types that comprise the lineage of cells produced when a stem cell differentiates into a particular cell type in vivo or ex vivo, and for identifying all of the genes that are conditionally expressed during the differentiation of a stem cell in vivo or ex vivo, as described below.

Other embodiments of the present invention uses recombinase to remove an exogenous DNA marker construct from purified differentiated cells that can then be used for subsequent rounds of genetic modification or for cell therapy. An example of such embodiments comprises inserting into the genomic DNA of a stem cell a marker DNA construct comprising two recombination sites flanking a marker DNA sequence encoding a detectable product, with expression of the marker DNA construct being placed under the control of a first conditional promoter. Another DNA expression cassette comprising a DNA sequence encoding a recombinase that recognizes the two recombination sites is also inserted into the genomic DNA of the same stem cell, the expression of this DNA cassette being placed under the control of a second conditional promoter. Either of the two DNA constructs can be inserted into an endogenous conditionally expressed gene at a site such that it is controlled by the endogenous promoter, or it can comprise an exogenous conditionally expressed promoter to which the DNA coding sequence is operably linked. In a useful embodiment, the marker DNA construct encoding a detectable product is inserted into an endogenous gene that is expressed in a cell type-specific manner in cells that have differentiated into a particular partially or fully differentiated cell type of interest, and the DNA sequence encoding a recombinase is operably linked to an exogenous inducible promoter. Methods for obtaining and using inducible promoters are well known (for example, see Ryding et al., 2001, supra). In the resulting system, the marker DNA construct encoding the detectable product, e.g., an antigenic cell surface protein, is expressed when the stem cells are induced to differentiate into the particular differentiated cell type of interest. In this embodiment, differentiated cells expressing the marker gene are isolated, e.g., using labeled antibodies that bind to the antigenic cell surface protein, and the isolated cells are then exposed to an agent or conditions that activate the promoter controlling expression of recombinase gene, and the newly synthesized recombinase excises the marker DNA construct encoding the detectable antigenic protein from the genomic DNA of the stem cells. Single or multiple cell surface antigens can be sequentially expressed on the cell surface to allow the isolation and propagation of populations of cells at particular stages of differentiation, with later activation of recombinase removing a single or multiple constructs to eliminate coding sequences that could elicit an immune response. Methods for obtaining and inducible promoters and the agents to which they respond are well-known in the art.

As described above, embodiments of the present invention also include known methods in which a nucleic acid expression construct comprising exogenous promoter sequences operably linked to a nucleic acid encoding a protein are introduced into the genomic DNA of gene trapped stem cells to effect expression of the construct in the cells. Since the expression construct is randomly inserted, its expression may suffer from possible position effects; however, the constructs can be made and used more easily than constructs required for introducing DNA by homologous recombination, and methods known in the art, including selection of 5′ and 3′ flanking sequences, and the use of insulator sequences, can eliminate some of the variability of expression.

Introduction of DNA constructs encoding elements of the Cre-lox and Flp-FRT systems, e.g., to permanently tag cell lines, is described above. Other genes of interest that can be co-expressed in identified cell clones include cell surface antigen to facilitate the purification of desired cells from a mixed population, and genes that function to influence a desired parameter in the cell's biochemistry, such as genes that block subsequent differentiation, or transcription factor that promote differentiation along particular pathways. For instance, a gene trapped stem cell containing a trapped gene that is expressed in early mesoderm can be genetically modified to express exogenous DNA sequences encoding myogenic transcription factors when it differentiates to the early mesoderm stage, to promote its differentiation and growth into myoblasts and myocytes. Those skilled in the art can devise many similar examples, given the state of knowledge in the art regarding differentiation promoting and inhibiting genes. The present invention further comprises genetically modifying stem cells by introducing DNA constructs comprising genes that function to promote growth of such cells by recognized growth pathways, and/or comprising genes from mammalian or non-mammalian species that encode mitogens that stimulate cell proliferation, e.g., the SV40-T-antigen. These genes can subsequently be removed by recombination as described above, e.g., using the Cre-lox system. When autologous stem cells are produced by nuclear transfer, parthenogenesis, or other technology that involves a specific cell line individualized to a particular patent or animal, expression cassettes containing such genes can be introduced in a single DNA construct, or they can be simultaneously introduced in separate DNA constructs, and the functional genes can then be removed by induced recombination. Such constructs can also be introduced in an artificial chromosome, and the functional genes or a critical feature of the chromosome can then be removed by induced recombination.

The embodiments and methods disclosed herein are not meant to be limiting, since those in the art will recognize that many useful variants of the disclosed methods can be devised, e.g., by varying the number and types of markers and/or promoters that are used, the number of endogenous genes that are marked, the method by which the constructs are inserted, etc.

I. Primary Screening

The present invention uses primary screening methods to identify mammalian genes that are conditionally expressed during differentiation of cells in vivo and ex vivo, or that show other unique and useful patterns of gene expression; e.g., conditional expression related to control of proliferation or cell survival.

A. Primary Screening with Gene Trapped Stem Cells:

Primary screening of the present invention can be performed efficiently by making libraries of gene trapped stem cells, exposing the cells to conditions in vivo or ex vivo that induce the cells to differentiate, identifying cells having marked genes that are conditionally expressed in the differentiating cells. The nucleotide sequences of the conditionally expressed genes can then be determined. A method for making gene trapped stem cells suitable for the present invention comprises introducing recombinant DNA constructs encoding an easily detected product (e.g., a specific mRNA, or a fluorescent or antigenic protein) into sites in genes of the stem cells' DNA to generate stem cells having marker DNA constructs that are under transcriptional control of the endogenous promoters of the genes in which they are inserted. For example, a library of gene trapped stem cells can be prepared by randomly inserting gene trap marker DNA constructs encoding a detectable marker such as GFP into the genomic DNA of a population of stem cells, and isolating and expanding the genetically modified stem cells as single cell clones. The latter step can be facilitated by including a DNA sequence encoding a selectable marker such as Neo^{r} (encoding neomycin resistance) in the marker DNA constructs for selection of the cells in which the marker DNA construct is expressed. Such DNA can be introduced by transfection, lipofection, microcell fusion, retroviral infection, injection, or other means known in the art. In preparing a library of gene trapped stem cells, it is straightforward to determine the “multiplicity of infection” (MOI) to generate a population of stem cell clones in which, in the population as a whole, either a selected fraction or all of the genes in the genome are tagged by a gene trap construct. In any given gene trapped stem cell in such a population, either a single or multiple genes in the genome are tagged by a gene trap construct. Generally a single insertion is desired per cell.

Large libraries of murine gene trapped ES cells having gene trap DNA markers randomly inserted in their genomes are presently being prepared and characterized, and the gene trapped ES cells are being made available to the public, as described above. As an efficient alternative to preparing novel gene trapped stem cell libraries, embodiments of the present invention use for the methods disclosed herein any libraries of gene trapped ES cells that are already known and available to those in the art.

Presently known methods (e.g., 5′-RACE) can be used to clone, isolate, and determine partial or complete nucleotide sequences of the genes in the cloned gene trapped stem cells in which the conditionally expressed marker DNA constructs are inserted.

The cloned stem cells are cultured ex vivo or in vivo in normal development, or as a chimera or a teratoma under conditions that induce their differentiation, and the cells are monitored to identify those stem cells having marker DNA constructs that are transcriptionally activated or repressed in the cells when the cells differentiate. In the case of humans, such differentiation in vivo as a chimera or an intact, developing embryo or fetus is considered by the inventors to be unethical and, although possible, is not considered to be part of this application.

Primary screening of a large number of different gene trapped stem cells can be performed ex vivo; for example, by plating the gene trapped stem cell in wells of multi-well culture plates, allowing them to spontaneously differentiate in vitro, and identifying the clones in which the marker genes are expressed.

In performing the screening assays of the invention, individual cells or individual groups of cells may be separated into any type of array apparatus or assembly of compartments that is convenient for systematically applying the test compounds and evaluating differentiation. For instance, the cells may be distributed into an apparatus comprising 10 to 100,000 different vessels or compartments, or for some embodiments 100 to 100,000 compartments, or for others 1000 to 10,000 compartments, or separate wells of one or more multi-well plates; e.g., 24-, 48-, or 96-well plates. A reference library of primary cells useful as controls can be freshly isolated and distributed in a similar array apparatus, or alternatively, frozen stock cells may be used. In distributing the cells into compartments, e.g., the wells of one or more multi-well plates, 1 to 10⁶ stem cells should be added per cm² of surface. Some stem cells, e.g., ES cells, require a minimum number of cells to survive, for such cells, 3 to 106 stem cells should be added per cm² of surface. Induction of differentiation by a given set of conditions occurs with a statistical probability; therefore, the more cells per well, the greater the likelihood that a cell in the well will be induced to differentiate. The plated gene trapped stem cells are exposed to various combinations of known and/or putative differentiation-inducing agents and are monitored to detect cells in which the level of marker gene expression increases or decreases relative to the level of expression in control cells. Suitable control cells include cloned cells cultured under conditions that do not induce differentiation, and differentiating cells having the marker DNA constructs functionally inserted into genes that are known be unexpressed during differentiation. Gene trapped stem cells having constitutively induced marker genes; e.g., marker genes that are co-regulated with endogenous genes recognized as having “housekeeping” functions, are also useful as control cells.

The cells can be automatically monitored continuously during the days and weeks following the induction of differentiation, to identify cells in which gene trap markers undergo a change in activity. For example, gene trapped stem cells in which DNA encoding GFP is inserted as a conditional marker can be monitored under a fluorescence microscope, the cells that fluoresce can be identified, and the time and intensity of marker expression can be recorded manually or robotically. Known methods (e.g., commercially available cell stains) and controls are also used to measure cell number, and to relate signal (e.g., fluorescence) intensity to total cell number, in order to normalize the results. Alternatively, gene trapped stem cells marked with a chromogenic marker such the lacZ gene can be monitored after exposure to compounds like fluorescein di-β-D-galactopyranoside (FDG), which form fluorescent products in the presence of β-galactosidase. Single cell clones having gene trap markers that show increased or repressed expression relative to control cells following induction of stem cell differentiation are then isolated for further analysis and characterization.

Primary screening of gene trapped stem cells can be performed in vivo by implanting gene trapped ES cells in immune-compromised animals, e.g., SCID mice, so that they form teratomas in vivo, and analyzing the teratomas histologically to identify tissues and cell types in which the marker genes are expressed.

Methods for culturing stem cells ex vivo or in vivo under conditions that induce their differentiation, and for monitoring the differentiating cells to identify the stem cells having marker DNA constructs that are transcriptionally activated or repressed in the cells when the cells differentiate, are discussed further below. Cloned lines of gene trapped stem cells that give rise to differentiating cells in which the marker DNA constructs are expressed are identified and selected for further analysis by secondary screening.

The primary screening methods of the present invention allow one to rapidly identify stem cell clones that have markers inserted in conditionally expressed genes, and to remove from the library the many clones that do not have such a marker. By monitoring the temporal pattern of expression of the genes, the present invention also allows one to identify markers that are useful for detecting and identifying cell types of lineages of the early pathways of cell differentiation in vitro, and for discriminating between alternative cell differentiation pathways. Such cell lines are of enormous utility in studying cell differentiation.

B. Determining the Nucleotide Sequences of the Conditionally Expressed Genes:

Once gene trapped stem cell clones having markers that are activated or repressed during differentiation are identified, known methods such as 5′ RACE can be used to sequence the genes in which the gene trap constructs are inserted. Analysis of the nucleotide sequences of genes that are conditionally expressed during differentiation can provide the nucleotide sequences of the mRNAs or other RNAs and the amino acid sequences of the proteins encoded by the genes, and these gene products can serve as markers for identifying the stage of differentiation of a cell, and as tags for purifying differentiated or partially differentiated cells from a population.

The nucleotide sequences of genes that are conditionally expressed during differentiation can also be analyzed to identify genes encoding proteins having amino acid sequences characteristic of cell surface receptors. As described below, secondary screening can then be performed to identify conditionally expressed genes encoding cell surface receptors that stimulate the growth of cells of a lineage formed by differentiation of stem cells into a particular cell type, or that serve as differentiation antigens whereby antibodies specific for such antigenic cell surface receptors can be used to isolate populations of cells at a selected stage of differentiation.

C. Representing the Data Obtained in Primary Screening:

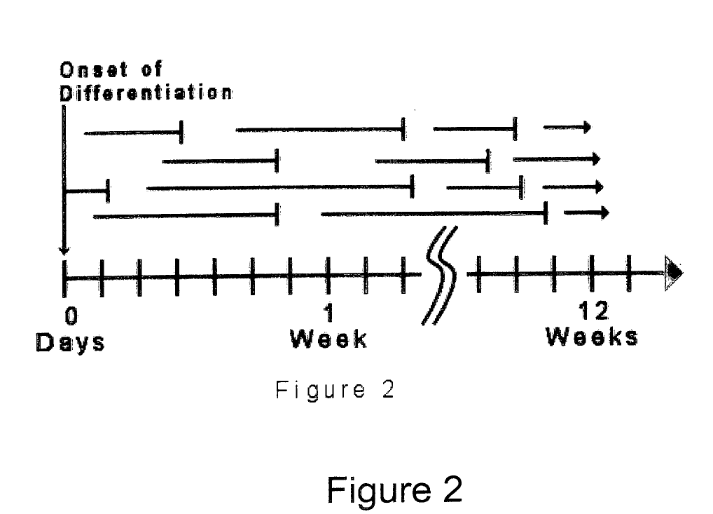

Data collected by the methods of the present invention showing the relative timing of transcriptional activation and/or repression of genes that are conditionally expressed during differentiation of stem cells in vivo or ex vivo can be graphically represented as a temporal gene expression map as shown in FIG. 2.

D. Primary Screening by Hybridization:

The primary screening steps of the present invention can also be performed effectively using available methods of molecular and cell biology. For example, using DNA microarray analysis or conventional hybridization assay methods and hybridization probes complementary to coding sequences identified by analysis of genomic DNA sequences and expressed sequence tags, mRNAs isolated from differentiating cells at various stages of differentiation in vivo or ex vivo can be analyzed quantitatively to identify genes that are conditionally expressed in differentiating cells.

The concentrations of hundreds or thousands of different mRNAs from different types of differentiating cells at various stages of differentiation in vivo or ex vivo can be analyzed quantitatively using DNA microarrays. Advances in rapid DNA sequencing and the determination of the nucleotide sequences of complete mammalian genomes are leading to production of DNA chips with arrays of hybridization probes complementary to most or all of the genes of a mammal. Large scale screening and quantitation of the mRNA profiles of cells differentiating into various cell types ex vivo or in tissues in vivo can be performed to detect and identify the conditional expression of a large number of genes that occurs during cell differentiation. Oligonucleotides complementary to DNA sequences of conditionally expressed genes identified by DNA microarray analysis or by conventional assay methods employing gel electrophoresis and membrane hybridization can be prepared for use as hybridization probes or as amplification primers to monitor the timing of the expression of these genes in differentiating cells, either in performing primary screening (in vitro or in vivo, e.g., with teratoma-forming stem cells) or in secondary screening in vivo during embryological and fetal development; e.g., by FISH.

E. Inducing Stem Cell Differentiation:

Methods are presently known for inducing stem cells to differentiate in vivo and ex vivo into a variety of different cell types. Additional details of these methods are discussed in the Background section above. In vivo methods for inducing stem cells to differentiate include injecting one or more embryonic stem cells into a blastocyst to form a chimeric embryo that is allowed to develop; fusing a stem cell with an enucleated oocyte to form a nuclear transfer unit (NTU), and culturing the NTU under conditions that result in generation of an embryo that is allowed to develop; and implanting one or more embryonic stem cells into an immune-compromised or a histocompatible host animal (e.g., a SCID mouse, or a syngeneic nuclear donor) and allowing teratomas comprising differentiated cells to form. In vitro methods for inducing stem cells to differentiate include culturing the stem cells in a monolayer, in suspension, or in three-dimensional matrices, alone or in co-culture with cells of a different type, and exposing them to one of many combinations of chemical, biological, and physical agents, including co-culture with one or more different types of cells, that are known to capable of induce or allow differentiation. As noted above, embryonic stem cells and embryonic germ cells can also be cultured in vitro by known methods under conditions that induce them to form embryoid bodies in which precursor or differentiated cells appear to arise randomly or spontaneously.

F. Conditionally Expressed Genes:

A gene that is conditionally expressed during differentiation is identified by detecting the activation or repression of its transcription in a cell of a lineage formed by differentiation of the stem cells into a particular cell type. As used herein, a conditionally expressed gene is one that is regulated such that its transcription is activated or repressed, or it is expressed at the same level but its product is stabilized or degraded at an accelerated rate, during a defined period of time in response to one or more external stimuli (chemical, biological, or physical) or environmental conditions to which the cell is exposed. To be conditionally expressed, the activation or repression of transcription of a gene need not be total. A gene is referred to herein as being conditionally expressed if an increase or decrease in its rate of transcription in a cell can be detected in response to exposure of the cell to one or more external stimuli or environmental conditions.

G. Detecting Conditionally Expressed Genes:

Various methods for detecting the activation or repression of transcription of genes that are conditionally expressed during differentiation of cells in vivo and in vitro are also presently available. Well-known methods include assaying to detect a change in the concentration of a mRNA or a polypeptide product encoded by a gene in the cell. For example, a change in the concentration of a mRNA in a cell can be detected by in situ hybridization with fluorescent hybridization probes (FISH) or with radiolabeled hybridization probes, or by using RT-PCR. Methods have also been developed for making and using arrays of nucleic acid hybridization probes bound to solid substrates (e.g., microarrays, “DNA chips”) that permit simultaneous detection and quantitation of the concentrations of many different mRNAs, isolated from a cell sample. Well-known methods for detecting a change in the concentration of a protein in a cell include methods that use antibodies that specifically bind to the protein; e.g., immunocytochemistry, western blotting, and the enzyme-linked immunosorbent assay (ELISA). Methods for using controls to distinguish signal from noise and ascertain that an increase or decrease in the rate of transcription of a gene has been detected are also well-known.

H. Isolating Gene Trapped Stem Cells:

Single cell clones of gene trapped stem cells, i.e., populations of cells derived from individual gene trapped stem cells, can be isolated by methods that are routine in the art. For example, gene trapped stem cells having expressed markers can be separated from cells with unexpressed markers by using gene trap constructs that contain a selectable marker such as Neo^{r}, a gene that confers resistance to the antibiotic neomycin. Cells expressing a marker DNA construct encoding a fluorescent product such as GFP may be purified from other cell types and from undifferentiated cells of the same type in a sample by fluorescence activated cell sorting (Odorico et al., 2001, Stem Cells 19(3): 193-204).

Methods for isolating stem cells expressing a marker DNA construct encoding an antigenic cell surface protein are described in Gay (U.S. Pat. No. 5,639,618), the contents of which are incorporated herein by reference. Automated systems also allow the destruction of unmarked cells (e.g., by laser) and the removal and subsequent culture of isolated marked cells. Using such methods, thousands of gene trapped stem cell lines of interest can be isolated as single cell clones. The selected gene trapped stem cells can then be expanded, cryopreserved, and used in further characterization of the genes in which the gene trap constructs are inserted, and in the secondary screening methods of the present invention that further elucidate the intra- and extracellular factors that regulate stem cell differentiation.

I. Secondary Screening

The primary screening methods of the present invention are used to identify, clone, and determine the nucleotide sequences of genes that are conditionally expressed in the differentiating cells of a mammal, and to determine the temporal pattern of their expression in the differentiating cells. The invention further provides secondary screening methods for monitoring the transcriptional activities of these conditionally expressed genes in specific types of differentiating cells in vitro and in vivo. These methods are used to monitor the expression of the conditionally expressed genes in vivo during the development of an embryonic, fetal, and adult mammal, to identify, for each of the partially or fully differentiated cell types of a mammal, a set of conditionally expressed genes that are transcriptionally activated at different times in cells of the lineage formed during the differentiation of stem cells into that cell type.

The secondary screening methods allow one to detect the sequential transcriptional regulatory events that occur over time when stem cells differentiate into each of the different cell types of a mammal, and to correlate the relative timing of these events with the generation of the different types of partially and fully differentiated cell types that arise during development.

The secondary screening methods of the invention are a powerful set of methods for identifying the set of conditionally expressed genes that are transcriptionally activated at different times in cells of the lineage formed during the differentiation of stem cells into any partially or fully differentiated cell type of a mammal. These methods are usually performed after identifying genes that are transcriptionally activated during differentiation of the stem cell into multiple cell types in vivo or ex vivo (primary screening), and comprise, for a particular partially or fully differentiated cell type of a mammal:

(a) inducing a population of the stem cells to differentiate into the particular cell type of interest; and

(b) assaying to detect multiple genes identified by primary screening that are transcriptionally activated in cells that are differentiating into the particular cell type of interest.

The stem cells are cultured in vivo and/or in vitro under conditions that induce their differentiation into a particular cell type of interest, and histological and molecular biological methods are used to:

(i) identify gene trapped stem cells having genes that are transcriptionally activated and/or repressed at different times in cells of a lineage formed by differentiation of the stem cells into the particular cell type of interest,

(ii) characterize the conditionally expressed genes in terms of the timing of their transcriptional activation and/or repression in vivo or ex vivo, and

(iii) describe and characterize the different type of cells and tissues in which the genes are expressed during their development and differentiation into the particular cell type of interest, including the cellular context (e.g. cell-cell contacts and the identities of neighboring cell types) of the expressing cells.

In monitoring the expression of conditionally expressed genes of stem cells differentiating in vivo, secondary screening further includes determining the physical locations of the differentiating cells in the body of a developing mammal at the time of transcriptional activation and/or repression of each gene that is identified as being conditionally expressed in the differentiating cells.

A. Screening to Detect Products of Conditionally Expressed Genes:

A central object of secondary screening is the detection of the transcriptional activation of conditionally expressed genes in cells that are differentiating into particular cell types. The expression of conditionally expressed genes in differentiating cells is detected using known methods. For example, the trapped genes identified by primary screening as being conditionally expressed in differentiating cells can be cloned and sequenced (e.g., using 5′-RACE ). Hybridization probes and amplification primers complementary to nucleotide sequences of the conditionally expressed genes can then be prepared and used to analyze mRNAs of cells that are differentiating into the particular cell type of interest, and detect and identify genes that are transcriptionally activated in the differentiating cells. As described above for primary screening, hybridization-based methods for detecting changes in the levels of expression of conditionally expressed genes include but are not limited to RT-PCR, DNA microarray analysis, and FISH.

Secondary screening can also be performed using methods that detect the proteins encoded by the conditionally expressed genes. Such methods can use assays that detect an enzymatic or biological activity of the protein or they can detect the proteins with antibodies that bind specifically to proteins encoded by the conditionally expressed genes. For example, antibodies that bind specifically to proteins encoded by conditionally expressed genes can also be labeled with detectable labels such as fluorescent groups and used in secondary screening to detect the differentiated state of cells grown in vitro or in vivo; for example, by immunocytochemistry. In a useful embodiment, cells that are at a particular stage of differentiation can be isolated, and the isolated cells or extracts thereof can be used to produce antibodies that bind specifically to a cell type-specific protein that is characteristically produced by the isolated, differentiating cells. Antibodies that bind to such “differentiation antigens” can then be used to detect cells producing the antigenic proteins as described above, or they can be used in non-invasive methods for isolating populations of differentiated cells of a particular cell type, as discussed below.

Fluorescent groups that emit light of different colors are commercially available (Panvera, LLC, Madison, Wis.), and can be used to label hybridization probes that bind to different conditionally expressed genes, or antibodies that bind to different antigens, and screening can be performed using mixtures of different probes or antibodies, each labeled with a fluor of a different color, for more rapid and efficient screening of the samples.

B. Screening with Gene Trapped Stem Cells:

The expression of conditionally expressed genes in cells that are differentiating into a particular cell type can also be detected and monitored using a library of cloned gene trapped stem cell lines containing marker DNA constructs that are co-expressed with the conditionally expressed genes identified by primary screening. Using gene trapped stem cells permits screening to be performed without first determining nucleotide sequences of the genes of interest, and allows one to use a single assay to detect expression of every gene of interest. Gene trapped stem cell clones used for secondary screening can each have a single trapped gene, or gene trapped stem cell clones can be used that have multiple trapped genes, each containing a marker DNA construct encoding the same or a different detectable product.

Gene trapped stem cells containing randomly inserted marker DNA constructs that are obtained by the above-described primary screening methods can be used in these methods. Alternatively, gene trapped stem cells useful for these methods can be made by inserting marker DNA constructs into targeted sites in the conditionally expressed genes by known methods utilizing homologous recombination so that they are transcriptionally regulated by the promoters of the genes in which they are inserted. Gene trap-like stem cells useful for the invention can also be made by identifying the promoter and enhancer DNA sequence elements of the conditionally expressed genes identified by primary screening that direct the cell type- and developmental stage-specific expression of the genes, and by transfecting stem cells with recombinant vectors comprising DNA marker constructs containing these promoter/enhancer elements operably linked to DNA sequences coding for detectable products. The exogenous promoter/enhancer elements used to direct cell type- and developmental stage-specific marker genes expression in these cells gene trap-like stem cells can be of the same species as the stem cells, or of a different species. These gene trap-like stem cells can generally be used in the same manner as true gene trapped stem cells in the screening methods and applications disclosed herein.

Marker DNA constructs encoding any detectable product can be used for the invention; examples include a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

C. Secondary Screening of Cells Differentiating Ex Vivo:

Secondary screening can be performed ex vivo; e.g., by culturing the stem cells in vitro in multi-well dishes under conditions that are known to induce differentiation of the stem cells into the particular cell type of interest. The stem cells can be cells without trapped genes, and the expression of genes identified in primary screening can be detected using known methods, e.g., with labeled hybridization probes or antibodies. Alternatively, gene trapped stem cells can be used in combination with assays to detect expression of the trapped genes. Methods are known for inducing stem cells to differentiate into a variety of different, specific cell types in vitro, as discussed above. Secondary screening can be performed in vitro by plating the stem cells in individual plates or in wells of multi-well culture plates, culturing them under conditions that induce differentiation into a particular cell type, and identifying the cells in which transcription of conditionally expressed genes are is activated. As in primary screening, individual cells or individual groups of cells can be plated and cultured in any type of array apparatus or assembly of compartments that is convenient for systematically applying differentiation-inducing agents or conditions and evaluating differentiation. The cells are monitored using known methods to detect the timing of transcriptional activation and to cytologically evaluate the cells in which expression is detected.

D. Secondary Screening of Cells Differentiating In Vivo:

Secondary screening can also be performed in vivo, using stem cells without trapped genes and detecting the expression of genes of interest using known hybridization-or antibody-based methods, or using gene trapped stem cells and assaying to detect expression of the trapped genes. The temporal pattern of expression of differentiation-specific genes observed during primary screening ex vivo is typically preserved in cells growing and differentiating in vivo. For example, if primary screening in vitro yields a murine gene trapped ES cell that activates a marker at day 18, then histological analysis performed at day 18 or later on a developing mouse generated from the gene trapped ES cell would be expected to reveal cells and tissues expressing the same marker.

The secondary screening methods described herein can be performed with mammals of any species, including humans; however, the disclosed in vivo screening methods are generally invasive or destructive of the subject mammal, and so are intended to be used only with non-human mammals. Methods that draw on results obtained using the disclosed primary and screening methods with stem cells of non-human mammals to identify the spatial and temporal patterns of gene expression occurring in differentiating human cell types of interest are described below.

(1) Screening In Vivo With Stem Cells that do not have Trapped Genes:

Secondary screening can be performed in vivo with wild type stem cells by systematically examining tissue sections of a developing non-human mammal histologically, and using assays to detect the expression of genes identified in primary screening. Tissues of an embryonic, fetal, and post-natal non-human mammal are sectioned and analyzed to identify specific partially differentiated or fully differentiated cell types of interest, and to detect the timing and location of transcriptional activation of the conditionally expressed genes in the cell types of interest. FISH is a powerful hybridization-based method that is well-suited for secondary screening to simultaneously detect and identify differentiating cell types in vivo. Nucleic acid hybridization probes complementary to DNA sequences of the conditionally expressed genes identified in the primary screening step can be labeled with fluorescent probes. Tissue sections of a developing non-human mammal, e.g., sections taken during the first 12 weeks of embryogenesis, are examined histologically and cytologically to identify specific partially differentiated or fully differentiated cell types of interest, and FISH is performed using the the labeled probes to detect the timing and location of transcriptional activation of the conditionally expressed genes in these cell types. Secondary screening in vivo can also be performed by implanting ES cells into an immunocompromised or otherwise histocompatible host animal, e.g., a SCID mouse, to form teratomas, that give rise to the differentiated cell types of interest, and examining tissue sections of these cells by the same methods described above for normal tissues. Performing secondary screening with cells and tissues of non-human primates is useful for identifying developmentally regulated genes associated with the differentiation of human cells.

(2) Screening In Vivo with Gene Trapped Stem Cells:

Once useful mammalian gene trapped stem cell clones are identified by primary screening as described above, they can also be used effectively in secondary screening methods. The expression of a trapped gene in a tissue containing gene trapped stem cells can be detected by assaying to detect the product encoded by the marker DNA construct inserted in the trapped gene.

Methods known in the art can be used to generate whole embryos, fetuses, or adult mammals from individual cloned stem cells. For example, gene trapped ES cells can be introduced into an early embryo to produce a chimeric embryo that gives rise to an adult having germ cells with the gene trapped genotype. From such an adult mouse, embryonic, fetal, and fully developed mice can be bred that are heterozygous or homozygous for the trapped gene.

Gene trapped stem cells can be tagged with a constitutively expressed reporter gene and introduced into an embryo to generate a chimeric embryo in which the tagged cells are distinguishable from the other cells, as described below. Such chimeric embryos, and the fetal and developed mammals that are generated therefrom, can be used effectively for secondary screening in vivo.

Embryonic and fully developed mammals having the genotype of a gene trapped stem cell can also be generated by nuclear transfer cloning using known methods comprising fusing the gene trapped stem cell with an enucleated oocyte and culturing the resulting nuclear transfer unit under conditions in which it activates and gives rise to a viable embryo. Embryonic, fetal, and developed mammals generated by these methods can also serve as sources from which gene trapped ES and other stem cell types can be isolated or derived using known methods.

Secondary screening in vivo can also be performed by implanting gene trapped ES cells into an immunocompromised or otherwise histocompatible host animal, e.g., a SCID mouse, to form teratomas that give rise to the differentiated cell types of interest, and examining tissue sections of these cells to detect cells in which the trapped genes are expressed.

Secondary screening in vivo of tissues containing gene trapped stem cells is performed in the same general manner as described above for screening with stem cells that do not have trapped genes, i.e., by systematically examining tissue sections of a developing non-human mammal histologically, and by assaying to detect the expression of genes identified in primary screening. As in screening with stem cells that do not have trapped genes, tissues of an embryonic, fetal, or post-natal non-human mammal are sectioned and analyzed to identify specific partially differentiated or fully differentiated cell types of interest, and to detect the timing and location of transcriptional activation of the conditionally expressed genes in the cell types of interest. The expression of trapped genes in tissues containing gene trapped stem cells is detected by assaying to detect the product(s) encoded by the marker DNA constructs inserted in the trapped genes.

E. Mapping Results of Secondary Screening to Branches of the Developmental Tree:

Determination of the temporal and spatial patterns of lineage-specific gene expression by the methods of the present invention will allow the assemblage of the temporal data points into temporal data points in a map of the developmental tree for any type of mammal. A developmental tree representing the differentiation of a totipotent stem cell into multiple different cell types is schematically depicted in FIG. 1. The linkage or correlation of the temporal regulation of the expression of developmentally regulated genes with the identities of the cell types in which the genes are expressed made possible by the present invention allows a meaningful mapping of the developmental tree, and permits the identification of cell clones that are useful in vitro markers for screening and monitoring conditions to manufacture specific desired cell types.

F. Verifying Results Obtained with Heterozygous Knockout Gene Trap Cells:

In making gene trapped stem cells, the insertion of marker DNA constructs into the trapped genes usually disrupts the targeted genes and prevents them from being expressed effectively. The trapped genes of the resulting gene trapped stem cells are referred to herein as “heterozygous knockout” genes, because only one allele of the gene in the cell is knocked out. In some cases, the undisrupted allele corresponding to a heterozygous knockout gene may fail to compensate for the loss of the disrupted allele, and stem cells having the heterozygous knockout gene may fail to differentiate normally. Accordingly the present invention includes methods for verifying that the spatial and temporal patterns of gene expression that are observed during the differentiation of gene trapped stem cells in vivo or in vitro also occur in cells without the heterozygous knockout genes. This can be done by making and using hybridization probes or amplification primers complementary to the conditionally expressed genes, and by analyzing mRNAs of cells that are differentiating into the particular cell type of interest, e.g., by FISH, as described above. Verification can also be carried out by making gene trapped stem cells that have marker DNA constructs inserted into the genes of interest at sites, such that the targeted genes are not disrupted and the marker DNAs are under control of the promoters of the genes in which they are inserted. Such gene trap cells can be made by presently available methods using homologous recombination. Secondary screening can then be performed using such gene trapped stem cells to verify that the patterns of gene expression observed using gene trapped stem cells with heterozygous knockouts also occur in normal cells.

### Lineage Marking of Cells used in Screening and Other Appications

It can be useful to perform secondary screening with stem cells that are genetically modified to have and express a marker DNA construct encoding a constitutive marker such as GFP. For example, if such stem cells are used to generate a chimeric embryo, they and their derivative cells can be detected in the developing embryo by monitoring for GFP fluorescence. Gene trapped stem cells having a marker gene that identifies a differentiated cell of interest can be supertransfected with a vector comprising a marker DNA construct encoding such a constitutive marker, and differentiated cells derived from these cells are useful in testing the efficacy and safety of cells administered for cell therapy in animal or human models. Detection of the cell type-identifying marker demonstrates to the investigator that the cell type of interest is present in the target tissue of interest, and the constitutive marker identifies the administered cells against the background of the host cells into which the test cells are administered.

### Uninodal vs. Multinodal Gene Trapped Stem Cell Lines

Some gene trapped lines will activate the marker gene in one lineage only. An example would be a marker that was activated only in cells en route to becoming a pancreatic islet cell. Such a marker is herein called a “uninodal” gene trap since the marker is activated in only a single cell lineage in the developmental tree. A “multinodal” gene trapped cell line is transcribed in multiple lineages. Although a multi-nodal marker is not in itself specific to a given lineage, data collected from monitoring the patterns of expression of combinations of multinodal gene trapped cell lines can, by means of logical deduction, lead to conclusions about specific cell lineages. For example, if a multinodal marker in cell line A is expressed during differentiation of the cells into heart, lung, and brain, and e.g., if a multinodal marker in cell line B is expressed during differentiation of the cells into eye, kidney, and brain:

| Cell Line A | Cell Line B |
|---|---|
| Heart | Eye |
| Lung | Kidney |
| Brain | Brain |

then, detection of the expression of both markers in cells of an unidentified lineage is evidence that the cells are differentiating as brain cells. Similarly, in vitro culture conditions that induce expression of both markers are likely to be conditions that induce stem cells to differentiate into brain cells.

### Identifying Multiple Genes that are Conditionally Expressed in Cells that are Differentiating into a Particular Cell Type

The secondary screening methods allow one to identify two or more genes that are transcriptionally activated at different times during differentiation of a stem cell into a particular cell type of interest. The cell type of interest may be either a partially differentiated cell type, or a fully or terminally differentiated cell type. Some of the genes identified by the invention are transcribed in multiple lineages; i.e., they are multinodal genes. This is often the case for genes that are expressed early in development. Other genes identified by the invention are transcribed only in the lineage of cells differentiating into the cell type of interest; i.e., are uninodal genes. Such genes are generally expressed only in the later stages of differentiation of a fully or terminally differentiated cell type.

The present invention comprises identifying two or more genes that are transcriptionally activated at different times during differentiation of the cells into the cell type of interest. For example, an embodiment of the invention comprises:

(i) determining the time period required for the stem cells to differentiate into the particular cell type of interest;

(ii) identifying at least one gene that is transcriptionally activated in cells differentiating into the cell type of interest before passage of 90% of the time period determined in step (i); and

(iii) identifying at least one gene other than the gene identified in step (i) that is specifically expressed in the cell type of interest; i.e., is expressed in a cell type-specific manner in the cell type of interest.

In this embodiment, step (ii) can comprise identifying at least one gene that is transcriptionally activated in cells differentiating into the particular cell type before passage of up to 5, 10, 15, 20, 25, 30, 35, 40, 45, 50, 55, 60, 65, 70, 75, 80, 85, 90, or 95% of the time period required for the stem cells to differentiate that is determined in step (i). The invention permits identification of all of the genes that are transcriptionally activated in cells that are differentiating into a particular cell type of interest. Hence, limits on the numbers of genes that can be identified are determined by Nature, and depend on the species and the identity of the cell type of interest. The invention comprises monitoring to detect the expression of marker DNA constructs in 1, 2, 3, 4, 5, 6, 7, 8, 9, 10, 11, 12, 13, 14, 15, 16, 17, 18, 19, 20, or more different genes that are transcriptionally activated in cells differentiating into the particular cell type before passage of up to 95% of the time period required for differentiation.

An embodiment of the invention also comprises:

(i) determining the time period required for the stem cell to differentiate into the particular cell type of interest;

(ii) dividing that time period into two or more time intervals of approximately equivalent duration; and

(iii) identifying two or more genes, each of which is transcriptionally activated during a different time interval in cells differentiating into the particular cell type. For example, the time period required for the stem cell to differentiate into the particular cell type can be divided into 2, 3, 4, 5, 6, 7, 8, 9, 10, 11, 12, 13, 14, 15, 16, 17, 18, 19, 20, 21, 22, 23, 24, 25, 26, 27, 28, 29, 30, or more approximately equivalent time intervals; and 2, 3, 4, 5, 6, 7, 8, 9, 10, 11, 12, 13, 14, 15, 16, 17, 18, 19, 20, 21, 22, 23, 24, 25, 26, 27, 28, 29, 30, or more genes can be identified, each of which is transcriptionally activated during a different time interval in cells differentiating into the particular cell type of interest. As used herein, “time intervals of approximately equivalent duration” and “approximately equivalent time intervals” are time intervals of the same duration, plus or minus 15% of the duration of the interval. This embodiment is useful in methods in which the sequential activation of transcription of the conditionally expressed genes is monitored to track the progress of the differentiation process; for example, in developing methods for inducing or controlling differentiation of the cells in vitro, or to select and isolate specific populations of partially differentiated cells.

### “Walking” the Developmental Tree—Using Cells with Multiple Gene Trap Markers

Until all of the differentiation-specific genes of a mammal are identified and their patterns of expression are characterized, there will be regions in the developmental tree for the mammal where no useful markers have been identified. Such regions may correspond to genetic loci that are unusually small or have chromatin structures that insertion of randomly inserted marker DNA constructs, so that they are rarely marked in a population of gene trapped stem cells. In such a case, when a gene (e.g., “gene X”) is identified as one that is conditionally expressed in the lineage of a particular cell type, the present invention provides a method that facilitates identifying some or all of the other developmentally regulated genes of the same lineage. The method comprises randomly inserting a marker DNA construct encoding a detectable marker into the genomic DNA of a population of stem cells, differentiating the cells into the particular cell type, and screening to detect differentiating cells having gene trap markers that are conditionally expressed in cells in which gene X is also expressed. The cells expressing gene X can be identified by any of the known means for detecting a cell expressing a gene of interest; e.g., FISH, in situ hybridization with radiolabeled probes and autoradiography, RT-PCR, or using labeled antibodies that bind to the product of gene X.

Alternatively, the stem cells can be genetically altered by inserting into gene X a DNA marker construct at a site such that it is under control of the gene X promoter, and these gene trapped stem cells can be used to make a library of gene trapped stem cells having randomly inserted marker DNA constructs for detection of additional genes expressed in the differentiating cells. The resulting gene trapped stem cells have at least two trapped genes—gene X, and a gene trapped by random insertion of the second DNA marker construct.

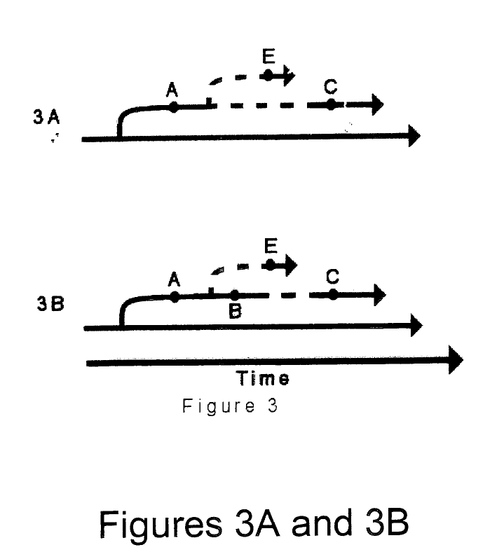

For example, a cloned gene trapped cell can be obtained that has a marker DNA construct encoding a detectable product such as GFP inserted in gene A that is expressed when the cell is induced to differentiate into a cell type of interest. The node or branch of the developmental tree corresponding to expression of gene A in the differentiating cell is shown in FIG. 3A. The dotted line extending from the node of gene A represents the temporal and spatial region of interest, and corresponds to genes that are transcriptionally activated in the same cell lineage as gene A subsequent to the induction of expression of gene A. To detect the conditional expression of additional genes in the lineage of gene A, a marker DNA construct encoding a second detectable marker distinguishable from that of gene A (e.g., another fluorescence gene) is randomly inserted into the DNA of the population of cells with the gene A trap. The cells are then differentiated to obtain cells that express the marker for gene A, and these cells are screened to detect cells the appearance of a novel marker B; that is, to detect differentiating clones in which both A and B are expressed, with B following A temporally. Clone B can then be secondarily screened in mice or teratomas and assigned to the missing region in the developmental tree as shown in FIG. 3B. The library of gene trapped stem cells expressing gene A can be screened further to identify additional clones having marked genes that are expressed after gene A in the differentiating cell lineage. The cloning and screening steps can be repeated again and again until all of the genes that are conditionally expressed during differentiation of the lineage of cells expressing gene A are identified. For example, a gene trap library made with cells in which genes A and B are trapped can be screened to identify cells containing three different marker DNA constructs that are expressed in the differentiating clones; these can be used to make a gene trap library for identifying cells containing four different marker DNA constructs that are expressed in the differentiating lineage; and so on. In practice, the same marker DNA construct can be used to mark two different genes in the same cell, if the timing of their expression is such that the transcriptional activation of one of the genes can be detected and distinguished from that of the other gene. The above-described approach can be used to trap and identify all or a large percentage of the genes in the genome of a mammal, to ensure that most or all of the cell type-specific and developmental stage-specific genes corresponding to nodes of the developmental tree are marked.

A similar approach to mapping the developmental tree uses recombinase- mediated lineage marking to identify all of the genes that are conditionally expressed in a cell lineage that is differentiating into a particular cell type. A method for using a recombinase-recombination site system such as Cre-lox or Flp-FRT for lineage marking is described above. Briefly, a DNA expression cassette encoding recombinase is inserted into a conditionally expressed gene of a stem cell so that it's expression is regulated by the promoter of the gene in which it is inserted. Another DNA expression cassette inserted into the DNA of the stem cell comprises (i) a consititutively activated promoter, (ii) a marker DNA sequence encoding a detectable product, and (iii) a nucleotide sequence flanked by two recombination sites that is inserted at a site (e.g., between the promoter and the marker coding sequence) such that it inhibits expression of the marker DNA. Transient expression of the gene in which the DNA encoding recombinase is inserted results in production of recombinase, which then excises the inhibitory nucleotide sequence from the marker DNA construct, allowing the constitutive promoter to activate transcription of the marker DNA encoding the detectable product. The marker DNA encoding the detectable product continues to be expressed under control of the constitutive promoter after the transiently activated gene in which the recombinase construct is inserted has become inactive. When a stem cell is lineage-marked in this manner, transient transcriptional activation of the marked gene (in which the recombinase construct is inserted) results in permanently activated expression of the marker DNA construct.

To identify all of the genes that are conditionally expressed in a cell lineage that is differentiating into a particular cell type, a marker DNA construct encoding a detectable marker can be inserted into a site in a gene of an ES cell (e.g., “gene Q”) that is developmentally regulated in the particular differentiated cell type of interest, so that it's expression is regulated by the promoter of gene Q. The node of the developmental tree corresponding to expression of the marked gene encoding the differentiation antigen is labeled Q in FIG. 4A. The dotted line extending from node Q to the stem cell represents the temporal and spatial region of interest, and corresponds to developmentally regulated genes that are transcriptionally activated in the lineage of cells differentiating into cells expressing the marked gene Q. The resulting gene trapped ES cell is isolated and expanded as a single-cell clone, and is used to generate a library of lineage-marked, gene trapped ES cells. This is done by randomly inserting into its DNA a lineage marker DNA construct comprising (i) a promoterless DNA cassette encoding recombinase, and (ii) a marker DNA cassette comprising a constitutive promoter, an inhibitory DNA sequence flanked by recombinase sites, and a marker DNA sequence encoding a detectable product that is distinguishable from that encoded by the marker in gene Q. The library of lineage-marked, gene-trapped cells is then differentiated to obtain cells that express the marker for gene Q, and is screened to detect cells that express both the marker inserted in gene Q, and the recombinase-dependent marker. The times at which the recombinase-dependent markers appear in the cells differentiating to express gene Q are recorded, and DNA sequences of the lineage-marked genes of these cells are cloned and sequenced. The lineage-marked genes expressed in cells of the differentiating lineage of interest can be assigned to the missing region in the developmental tree, in the temporal order in which they are expressed, as shown in FIG. 4B. Of course, this method does not necessarily have to be performed using gene trapped stem cells that have a marker DNA construct inserted into a gene such as gene Q that is expressed in the differentiating cells. For example, if gene Q encodes a cell type-specific mRNA or a differentiation antigen of the cell type of interest, the library of lineage-marked, gene-trapped cells can be made using normal, unmarked stem cells. The lineage-marked, gene-trapped stem cells are induced to differentiate into the cell type of interest, and the resulting cells can be screened to detect cells that have the cell type-specific mRNA or differentiation antigen and also express the recombinase-dependent marker. The cloned stem cells that express both markers can then be secondarily screened in mice or teratomas to determine their temporal and spatial patterns of expression in vivo. The method can be performed for each cell type of interest, to identify the developmentally regulated genes that are expressed in the cell lineages that differentiate into each cell type of a mammal.

### Mapping Branches of the Human Developmental Tree

Secondary screening by monitoring the expression of conditionally expressed genes in developing embryonic, fetal, and adult mammals is generally destructive of the subject, and so cannot be used directly to identify the spatial and temporal patterns of expression of genes in differentiating cells of humans. Instead, various “bootstrapping” approaches are taken to map developmentally regulated genes to branches of the human developmental tree leading to differentiation of cell types of interest. These methods have in common the steps of (a) identifying developmentally regulated genes that could possibly be expressed in human cells that are differentiating into a particular cell type of interest, and (b) inducing human stem cells to differentiate into the cell type of interest, and assaying to determine if the candidate genes are transcriptionally activated in the differentiating human cells.

A. Screening to Detect Human Homologs of Developmentally Regulated Genes of Non-human Mammals.

Genes that regulate development and differentiation tend to be highly conserved in mammals of different species. Therefore, genes that are likely to be developmentally regulated in differentiating human cells can be identified by first using the above-described primary and secondary screening methods to identify genes that are conditionally expressed in cells of a non-human mammal that are differentiating into specific cell types of interest. Screening can then be performed by culturing human cells under conditions that induce their differentiation into a cell type of interest, and monitoring to detect the expression of human genes that are homologous to genes that are developmentally regulated in non-human cells that are differentiating into the cell type of interest. Developmentally regulated genes of any species of non-human mammal can be used to identify human homologs in this manner. For example, publicly available libraries of well-characterized murine gene trapped stem cells can be subjected to primary and secondary screening to identify developmentally regulated genes that are expressed in murine cells differentiating into one or more specific cell types of interest, and to determine their temporal and spatial patterns of expression in embryonic and fetal mice, as described above. Human genes homologous to these developmentally regulated genes can then be identified, and the temporal and spatial patterns of their expression in human cells differentiating into the same specific cell types of interest can be monitored.

The nucleotide sequences and patterns of expression of developmentally regulated genes of non-human primate species are very similar to those of human developmentally regulated genes. Accordingly, primary and secondary screening can be performed to identify developmentally regulated genes that are expressed in cells of a non-human primate that are differentiating into specific cell types of interest, and to determine the temporal and spatial patterns of expression of these genes in vivo. The patterns of expression of the homologous human genes can then be monitored in human cells differentiating into the same cell types ex vivo or as cells of teratomas growing in vivo, as described below. The screening can be performed to monitor the expression of human genes homologous to conditionally expressed genes of any non-human primate, e.g., common chimpanzee (Pan troglodytes); pigmy chimpanzee (Pan paniscus); gorilla (Gorilla gorilla); orangutan (Pongo pygmaeus); baboon (Papio hamadryas); gibbon (Hylobates lar); green monkey (Cercopithecus aethiops), tamarin (Saguinus oedipus); and other species of Old and New World monkeys. Cynomologous monkeys (Macaca fascicularis) and Rhesus monkeys (Macaca mulatta) are examples of well-characterized if monkey species that can be used in these screening methods.

Nucleotide sequences of developmentally regulated genes of non-human mammals that are expressed in differentiating cell types of interest can be determined by known methods (e.g., using 5-RACE), as discussed above; and the homologous human genes can then be identified, cloned, and sequenced, using well-known methods of molecular biology. The homologous human genes can be also identified by comparing the nucleotide sequences of the non-human genes to publicly available databases of human genomic DNA sequences. Computer programs that can efficiently perform such DNA sequence comparisons and identify the homologous genes are freely available.

Secondary screening to analyze the differentiation pathways of human cells can be carried out by inducing human stem cells to differentiate ex vivo and/or in stem cell-derived teratomas growing in vivo into particular cell types of interest, and monitoring the differentiating cells to detect the transcriptional activation of human genes that are developmentally regulated in the differentiating cells in a manner similar to that of their non-human homologs. Methods are presently known, and new methods are being developed, for inducing stem cells to differentiate ex vivo and in vitro into a growing number of specific cell types, as described above. These methods include culturing the stem cells in a monolayer or in suspension, and exposing them to various combinations of chemical, biological, and physical agents that are known to induce or allow their differentiation. Biological inducers of differentiation that can be used include cells of one or more different types that, when co-cultured with the target cells, induce their differentiation (discussed below). Known methods for inducing human cells to differentiate ex vivo also include culturing human ES cells and/or embryonic germ cells under conditions that induce them to form three-dimensional cellular structures such as artificial tissues and embryoid bodies that give rise to specific types of partially differentiated (e.g., precursor) and fully differentiated cells, also as discussed above. Methods are also available for inducing human ES cells to differentiate and generate specific cell types in vivo by implanting them into an immune-compromised or otherwise histocompatible host animal (e.g., a SCID mouse), and allowing the implanted stem cells to form teratomas that give rise to the specific differentiated cell types. The transcriptional activation of human genes homologous to non-human developmentally regulated genes in the differentiating cells is monitored and detected using known methods; e.g., using hybridization-based assays, or by assaying to detect the proteins encoded by the genes. When a developmentally regulated gene encodes a protein that is known to be expressed in cells of a particular type, the lineage or specific cell type in which the gene is expressed can be determined by identifying the protein that it encodes. In case a method for inducing human cells to differentiate into a particular cell type is not known or available, screening methods that can be used to determine combinations of chemical, biological, and physical agents that induce or allow the cells to differentiate into the cell type of interest are described in the forementioned co-owned and co-pending PCT Application No. PCT/US02/26945, filed Aug. 26, 2002, the contents of which are incorporated herein by reference in their entirety.

Changes in the level of expression of human genes homologous to conditionally expressed genes of non-human mammals can be monitored by any known means for detecting and quantitating gene expression. For example, known methods can be used to analyze mRNAs of specific differentiating cell types using a DNA chip with a microarray of hybridization probes complementary to the genes of interest to quantitatively detect the expression of the genes in the differentiating cells. Alternatively, labeled hybridization probes complementary to the genes of interest, or labeled antibodies that bind specifically to proteins encoded by the genes of interest, can be used to quantitatively detect the expression of these genes in the differentiating human cells. In situ histological analysis of the differentiating cells can be combining with the use of such labeled hybridization probes and/or labeled antibodies to quantitatively detect gene expression (e.g., FISH) as well as observe the cellular context (e.g., cell-cell contacts) of the differentiating cells in which conditional expression occurs.

B. Screening Using Human Gene Trapped Stem Cells.

Genes that are developmentally regulated in differentiating human cells can also be identified using human gene trapped stem cells in primary and secondary screening methods described above.

For example, a library of clones of human gene trapped ES cells having marker DNA constructs randomly inserted in their genomic DNA can be screened by culturing the ES cells ex vivo under conditions that are known to induce their differentiation, and monitoring to detect the transcriptional activation of the marker DNA constructs in the differentiating cells. Alternatively, human genes that are homologous to developmentally regulated genes of a non-human mammal can be identified as described above, and human stem cells can be genetically modified to express marker DNA constructs when these human genes are transcriptionally activated.

For example, human stem cells can be stably transfected with DNA vectors containing marker DNA constructs comprising exogenous cell type-specific or developmental stage-specific promoter/enhancer sequences that are transcriptionally activated at approximately the same times as conditionally expressed genes. Cell type-specific or developmental stage-specific promoter/enhancer sequences of non-human mammals have been isolated and shown to operate in human cells in cell type-specific or developmental stage-specific fashion, and such known sequences can be used to direct the conditional expression of marker genes in differentiating human cells for the present invention. The cell type-specific and/or developmental stage-specific promoter/enhancer sequences of the homologous human genes can also be isolated by known methods and used to make DNA marker constructs that are conditionally expressed in differentiating human cells at approximately the same times as the corresponding endogenous genes.

Human screening can also be performed using human gene trapped stem cells having marker DNA constructs inserted into the genes that are homologous to genes of non-human mammals that are conditionally expressed in differentiating cells. For example, marker DNA constructs encoding a detectable product can be targeted into sites in the human genes of interest by known methods utilizing homologous recombination so that they are under control of the promoters of the genes in which they are inserted, as discussed above. Such marker constructs can be inserted at sites such that they do not inhibit expression of the endogenous genes, or at sites chosen to disrupt the endogenous genes and prevent their expression.

Individual human gene trapped stem cells produced as described above can be isolated and expanded into single cell clones. The cloned human stem cells can then be cultured ex vivo and as teratoma cells growing in vivo to differentiate into specific cell types of interest, and the differentiating cells can be monitored to detect the transcriptional activation of the marked genes, also as described above. Such methods are not useful for identifying trapped genes that are developmentally regulated in human cells that are differentiating into the specific cell types of interest; and for determining the temporal pattern of expression of these genes in the differentiating cells.

The above-described methods for screening to identify and determine the patterns of expression of developmentally regulated genes expressed in differentiating human cells do not include the histological evaluations of embryonic and fetal gene expression that are performed with non-human mammals. Therefore, the spatial patterns of expression in developing human embryos and fetuses of the developmentally regulated genes identified by screening must be inferred from the patterns of expression of the homologous genes observed in developing non-human primates and other mammals. The above-described screening methods identify specific developmentally regulated genes that are expressed in human cells that are differentiating into specific cell types of interest, and determine the temporal pattern of expression of these genes in the differentiating cells. This information can be used in methods for determining new methods for inducing stem cells to differentiate into the specific cell types, for monitoring the progress of differentiating cells, isolating purified populations of cells that are at a particular stage of differentiation, and for preparing cells that are differentiated into specific cell types in which key genes of interest are inactivated as homozygous knockouts, as discussed below.

### Applications Utilizing Temporal Maps of Expression of Developmentally Regulated Genes

The present invention further provides methods that use temporal maps of genetic regulatory events occurring during differentiation of specific cell types obtained by the above-described screening methods. The temporal maps provided by the present invention can be used in methods for identifying sets of chemical, biological and physical conditions that induce the differentiation of stem cells into the numerous specific cell types of a mammal. The invention also provides methods for identifying cell differentiation markers that are specific for each stage of differentiation leading to a specific partially or fully differentiated mammalian cell type. This information can be used to isolate purified populations of cells that have differentiated into a particular partially or fully differentiated mammalian cell type. Such partially and fully differentiated cell populations can be used to prepare antibodies against differentiation antigens that are characteristic of specific cell types, and are useful for basic research, drug discovery and testing, toxicological studies, animal testing of cell therapy, and for therapeutic transplant to human and non-human patients in need of such transplant.

### Identifying Conditions that Influence the Differentiation of Stem Cells Ex Vivo

The present invention identifies a set of developmentally regulated genes that are conditionally expressed in cells differentiating into any partially or fully differentiated cell type of interest, and determines the relative timing of transcriptional activation of these genes in the differentiating cells. This information can be used to monitor the progressive differentiation of stem cells ex vivo in the presence of various combinations of chemical, biological, and physical agents that are capable of influencing the pathway of differentiation taken by the cells. Accordingly, new methods for inducing stem cells to differentiate ex vivo into a particular cell type can be identified by culturing the stem cells in vitro in the presence of many different combinations of chemical, biological, and physical agents that might possibly influence differentiation, and monitoring to detect expression of the developmentally regulated genes that are expressed in cells differentiating into the cell type of interest. As stated above, such screening methods are described in the aforementioned co-owned and co-pending PCT Application No. PCT/US02/26945, filed Aug. 26, 2002 (incorporated herein). Combinations of conditions that activate transcription of developmentally regulated genes in a temporal pattern associated with differentiation of the stem cells into a cell type of interest can be identified and subjected to further screening and refinement to develop an efficient ex vivo method for generating a cell type of interest.

### Monitoring the Differentiation of Stem Cells Ex Vivo into Specific Cell Types of Interest

Stem cells can be induced to differentiate in vitro into some cell types by exposure to a single combination of chemical, biological, and physical agents. When the stem cells are cultured under such conditions, each of the developmentally regulated genes associated with differentiation of the cell type of interest is expressed in succession, and detection of the transcriptional activation of these genes in the differentiating cells provides a series of “mileposts” for monitoring the progress of the differentiation process. The expression of developmentally regulated genes in the differentiating cells can be monitored by assaying to detect changes in the concentrations of mRNAs or proteins encoded by the genes, or by using stem cells containing marker DNA constructs that are co-expressed with the developmentally regulated genes, as discussed above. The invention thus provides an efficient means for monitoring the progressive differentiation of stem cells in vitro in order to precisely determine when the cells have reached a particular stage of partial differentiation, e.g., into a pluripotent stem cell such as a multipotential progenitor cell, or a progenitor cell committed to giving rise to one or a few fully differentiated cells, or into a fully differentiated cell type.

In some cases, the stem cells must be exposed successively to two or more different combinations of chemical, biological, and physical agents, in order to induce their differentiation into a cell type of interest. For example, when stem cells must be cultured successively under two sets of conditions to induce their differentiation into a cell type of interest, the initial combination of culture conditions may induce their differentiation into cells of a partially differentiated cell type, e.g., a pluripotent stem cell or committed precursor cell, and the final set of culture conditions may then induce their differentiation into a fully differentiated cell type of interest. By monitoring to detect the serial activation of expression of developmentally regulated genes in the differentiating cells, one can efficiently monitor the progress of stem cell differentiation to determine when a significant fraction of the cells have differentiated into the partially differentiated cell type produced the initial combination of culture conditions, and into the fully differentiated cell type of interest produced by culturing under the final set of conditions.

As described below, the present invention also provides methods for isolating purified populations of cells that are partially or fully differentiated into a specific cell type of interest. Using the methods described herein, one can -

(i) culture stem cells ex vivo under an initial set of conditions to induce their differentiation into a partially differentiated cell type,

(ii) detect when a significant fraction of the cultured cells are differentiated into a specific, partially differentiated cell type,

(iii) isolate a purified population of cells of the partially differentiated cell type,

(iv) culture the isolated population of cells ex vivo under another set of conditions that induces their differentiation into a fully differentiated cell type of interest,

(v) detect when a significant fraction of the cultured cells are differentiated into the fully differentiated cell type, and

(vi) isolate a purified population of the fully differentiated cells.

Of course, those in the art will recognize that many useful variants and combinations of these methods can be devised, e.g., by varying the methods used to detect expression of the developmentally regulated genes associated with the differentiation of the cell type of interest, the number of successive sets of cell culture conditions used to induce differentiation, or by choosing to change cell culture conditions without first isolating the partially differentiated cells.

### Purifying Populations of Differentiated Cells of Particular Types

The temporal maps provided by the present invention identify developmentally regulated genes that are expressed in cells that are differentiating into a specific partially or fully differentiated mammalian cell type, and the relative timing of the transcriptional activation of these genes in the differentiating cells. This information can be used to identify or generate cell differentiation markers in the differentiating cells that are specific for each stage of differentiation leading to a specific partially or fully differentiated mammalian cell type of interest. One can then isolate purified populations of cells that have differentiated to the particular stage at which a selected differentiation marker is expressed. The present invention thus provides methods for producing and isolating pure populations of cells that have reached a particular stage of differentiation; e.g., different types of pluripotent stem cells, committed progenitor cells, or fully differentiated cells of a human or non-human mammal. The purified, homogeneous populations of cells that are partially or fully differentiated into particular cell types that are the produced by these methods can be used for basic biological and biomedical research, drug discovery and testing, toxicological testing and sensing, and for animal pre-testing and clinical testing of cell therapy methodologies.

Cells that are differentiating into a particular partially or fully differentiated cell type of interest can be isolated at any stage of differentiation at which a developmentally regulated gene associated with differentiation into a cell type of interest is expressed. In one embodiment, stem cells are genetically modified by inserting into their genomic DNA a marker DNA construct encoding a detectable marker that is co-expressed with a developmentally regulated gene that is expressed in cells at the stage to be purified. A population of cells at the desired stage of differentiation can then be purified by inducing differentiation, allowing the cells to reach the desired stage of differentiation, and isolating cells that have the detectable marker from the cells that do not have the marker.

Methods for preparing genetically modified stem cells having a marker DNA construct encoding a detectable marker that is co-expressed with a developmentally regulated gene in cells differentiating into a particular cell are presently known, as discussed above. For example, a library of cloned gene trapped stem cells having marker DNA constructs randomly inserted in their genomic DNA can be screened by the primary and secondary screening methods described above to obtain a set of cloned gene trapped stem cells having marker DNA constructs that are co-expressed with developmentally regulated genes at various stages of differentiation of the cells into a cell type of interest.

Alternatively, the nucleotide sequence of a developmentally regulated gene that is expressed in the differentiating cells at the desired stage of differentiation can be determined, and the stem cells can be genetically modified by inserting into their genomic DNA a marker DNA construct that is co-expressed in the differentiating cells at the same stage of differentiation as the target developmentally regulated gene.

This can be done by using known methods utilizing homologous recombination to make gene trapped stem cells having a marker DNA construct encoding a detectable product inserted into a developmentally regulated gene that is expressed in the differentiating cells at the desired stage of differentiation. The marker DNA construct can be inserted in a site in the targeted gene so that it is under control of the promoter of the gene in which it is inserted. As discussed above, such a marker DNA construct can be inserted at a site selected so that it disrupts and/or inhibits expression of the endogenous gene, or at a site chosen so that expression of the endogenous gene is unaffected.

The stem cells can also be stably transfected with DNA vectors containing marker-specific or developmental stage-specific DNA constructs comprising exogenous cell type promoter/enhancer sequences that are transcriptionally activated at approximately the same times as conditionally expressed genes. Such cell type-specific or developmental stage-specific promoter/enhancer sequences and the stem cells in which they are used can be of the same or of different mammalian species. As used herein in this context, genes that are expressed “at approximately the same time” are genes that are transcriptionally activated at times sufficiently close to one another that isolation of cells expressing one gene yields a population of cells at essentially the same stage of differentiation as would be obtained by isolating cells expressing the other gene. The time period between the times at which two genes expressed “at approximately the same time” are transcriptionally activated is thus dependent on the rate of differentiation of the cells at the differentiation stage of interest. A useful marker DNA construct can contain two or more separate expression cassettes, one containing an exogenous developmentally regulated promoter operably linked to a DNA sequence encoding a detectable marker for identification of cells at the desired stage of differentiation, and one or more additional cassettes containing promoter and marker DNA combinations selected for specific purposes. For example, the marker DNA construct can comprise a first cassette containing an exogenous developmentally regulated promoter operably linked to a DNA sequence encoding Neo^{r}, and a second cassette containing a different exogenous developmentally regulated promoter operably linked to a DNA sequence encoding Hygro^{r}; with the promoter in the first cassette directing expression at a specific, early stage of differentiation, and the promoter in the first cassette directing expression at a specific, later stage of differentiation. Stem cells transfected with such a construct can be induced to differentiate, and a population of cells that has differentiated to the early stage of differentiation can be purified; the cells can then be expanded and induced to differentiate further, and a population of cells that has differentiated to the later stage of differentiation can also be isolated. Similar results can be obtained with cloned gene trapped stem cells that have marker DNA constructs inserted into two or more developmentally regulated genes that are expressed in cells differentiating into the cell type of interest.

A marker DNA construct containing multiple separately regulated expression cassettes can also be used to effect cell type-specific expression of a detectable marker DNA in cells that have differentiated to a differentiation stage of interest, and constitutive expression of a detectable marker that “tags” the cells; e.g., for detecting the cells in vivo after implanting them for animal testing of cell therapy. A marker DNA construct containing multiple separately regulated expression cassettes is also useful for effecting recombinase-mediated activation or inhibition of transcription of a DNA expression cassette as described above, or to effect recombinase-mediated excision of exogenous DNA from a cell to generate a genetically “wild type” cell that can be used for cell therapy as described below.

Bicistronic or multicistronic marker DNA constructs encoding a mRNA having two or more separately translated open reading frames can also be used in making the above-described genetically modified cells, in order to effect expression of two or more DNA expression cassettes in the marked cells. Methods for making such constructs utilizing DNA sequences encoding IRES elements are described above. For example, a gene trapped stem cell can be made using a bicistronic marker DNA construct with cistrons encoding GFP and Hygro^{r}; the GFP cistron allows the identification of clones in which the marker is expressed by detection of GFP fluorescence, and the Hygro^{r} cistron allows purification of the marked clones by using hygromycin to selectively eliminate the unmarked cells.

Known methods can be used to purify differentiating cells in which a marker DNA construct is expressed. Cells expressing a selectable marker such as Neo^{r} can be cultured in medium containing a selective agent such as G418 that kills cells in which the selectable marker is not expressed. Cells expressing an optically detectable marker can be separated from other cells by flow cytometry and automated cell sorting. For example, cells expressing a marker DNA encoding a fluorescent protein such as GFP, or an enzyme that converts a fluorogenic substrate into a fluorescent product such as β-galactosidase or β-lactamase, can be purified by fluorescence-activated cell sorting (FACS). Cells expressing a marker that is a cell surface antigen (i.e., a differentiation antigen) can be purified using antibodies that bind specifically to the cell surface antigen. For example, antibodies labeled with fluorescent groups can be bound to the cell surface antigen, and the labeled cells can be separated by FACS, or the cells having the cell surface antigen can be purified by known affinity-based methods. Expression vectors containing marker DNA constructs encoding cell surface antigens that facilitate cell purification are commercially available. For example, vectors are available that encode a trypsin-resistant, antigenic H-2K protein, and a truncated antigenic CD4 protein (Militenyi Biotech, Auburn, Calif.). Cells expressing these antigenic cell surface proteins can be sorted or purified by commercially available affinity columns or microbeads, or by other affinity separation techniques.

Purification using a selectable marker may be desirable because it allows isolation of the marked cells without having to remove them from their dish. The latter is a step that involves trypsinizing the cells, and is called for when cell sorting is used to purify the marked cells. Trypsinizing the differentiated or partially differentiated cells may alter the differentiation state of the cells. Trypsination may also remove cell surface antigens from the cells being purified, a result that is undesirable when the purified cells are to be used as immunogens to elicit production of cell type-specific differentiation antigens (discussed below), or when a cell surface antigen is used as the basis for cell sorting.

When purifying differentiated cells that are to be used for testing or practicing cell therapy, it may be desirable to obtain ‘wild-type’ cells that do not contain exogenous marker DNA constructs introduced through genetic modification. Exogenous marker DNA constructs can be removed from purified differentiated cells having such constructs by using an inducible DNA expression cassette encoding recombinase. The stem cells are genetically modified by insertion into their DNA a marker DNA construct having a first expression cassette comprising two recombination sites flanking a marker DNA sequence encoding a detectable product, and having a second expression cassette comprising an inducible promoter operably linked to a DNA sequence encoding a recombinase that recognizes the two recombination sites. Culturing the purified, differentiated cells in the presence of an inducer that activates the inducible promoter results in synthesis of the recombinase, which then excises the marker DNA sequence from the genomic DNA of the differentiated cells. The resulting cells will still contain an exogenous expression cassette encoding recombinase. A marker DNA construct comprising recombination sites flanking both expression cassettes can be used to effect excision of both exogenous expression constructs from the purified cells. Methods for making and using DNA marker constructs to effect recombinase-mediated excision of exogenous DNA from a cell are described in detail above.

“Wild-type” cells that have differentiated to a particular stage of differentiation can be purified for cell therapy using antibodies specific for an antigenic cell surface protein that is characteristically expressed in the differentiated cells in a developmental stage-specific manner. Methods for identifying such cellular differentiation antigens, and for making antibodies that bind specifically to such antigens that can be used to isolate the cells at a desired stage of differentiation, are described below.

### Isolating Differentiated Cells and Using them to Make Antibodies that Specifically Bind to Differentiation Antigens of the Purified Differentiated Cells

In preparing differentiated cells suitable for transplant therapy to be used for animal testing and clinical development, it is important to obtain cells having minimal genetic modification, because the products of exogenous genes (e.g., Neo^{r}) could elicit an immune response leading to rejection of the transplant, and because therapy based on the use of normal, unmodified cells is likely to have more predictable effects and so receive regulatory approval more quickly than therapy using genetic modified cells.

The expression of cell plasma membrane components changes during differentiation. These antigens are called differentiation antigens (Boyse and Old, 1969 Ann. Rev. Genet. 3:269-289). They can be identified by monoclonal antibodies, and cells that have such antigens can be sorted from cells that don't (positive selection). Monoclonal or polyclonal antibodies that specifically bind such differentiation antigens can be used to purify the cells having the antigens. Alternatively, the cells in a sample can be subjected to selection that removes or kills (undesired) cells that do not have the differentiation antigen, leaving only the cells that do have the antigen (negative selection). The generation of monoclonal antibodies to Drosophila retinal differentiation antigens is described in Zipursky et al, 1984, Cell, 36:15-26.

The methods of the present invention can be used to induce genetically modified (e.g., gene trap) stem cells to differentiate ex vivo into partially or fully differentiated cell types that produce one or more differentiation antigens specific for the cell type of interest, and then to isolate populations of the differentiated cells having such cell typespecific differentiation antigens. A purified population of such genetically modified, cells of a particular partially or fully differentiated cell type having a cell type-specific differentiation antigen is then used to produce antibodies that specifically bind to the differentiation antigen.

A purified population of genetically unmodified, differentiated cells having a cell type-specific differentiation antigen can then be prepared by culturing “wild-type,” i.e., normal, genetically unmodified, stem cells ex vivo under conditions in which they differentiate into the cell type having the differentiation antigen, and purifying the cells having the differentiation antigen using known affinity methods employing the antibodies that specifically bind to the differentiation antigen.

Accordingly, the invention provides methods for producing antibodies that bind specifically to a differentiation antigen of a particular differentiated or partially differentiated cell type of interest. Useful embodiments of such method comprise:

(a) obtaining a stem cell having inserted in its genomic DNA a marker DNA construct comprising a nucleotide sequence encoding a detectable product that is transcriptionally activated in cells of a particular differentiated or partially differentiated cell type having a differentiation antigen specific for said cell type;

(b) inducing the stem cells to differentiate into the particular differentiated or partially differentiated cell type;

(c) monitoring the differentiating stem cells to detect activation of transcription of the marker DNA construct in the cells;

(d) purifying cells having a transcriptionally activated marker DNA construct;

(e) using the purified cells or an extract thereof as an immunogen to elicit production of an antibody that binds specifically to the differentiation antigen specific for said cell type.

Useful polyclonal antibodies against such differentiation antigens can be produced by known means; e.g., by injecting whole cells or extracts of cells having a differentiation antigen into an animal, and obtaining antiserum from the animal that contains the antibodies. Useful monoclonal antibodies against such differentiation antigens can also be produced by known means comprising generating a hybridoma that produces the monoclonal antibodies. Antibodies against such differentiation antigens can also be produced by analyzing the nucleotide sequences of marked genes to identify marked genes that encode proteins having structures typical of antigenic cell surface proteins, expressing the genes in a cell-free system or in transfected cells, purifying the differentiation antigen, and using it to elicit polyclonal or monoclonal antibodies by known means.

Methods for obtaining a stem cell having inserted in its genomic DNA a marker DNA construct comprising a nucleotide sequence encoding a detectable product that is transcriptionally activated in cells of a particular differentiated or partially differentiated cell type are described. Stem cells having a marker DNA construct that is transcriptionally activated in cells of a particular differentiated or partially differentiated cell type of interest having a differentiation antigen are identified or produced using known methods.

The marker DNA construct of such genetically modified stem cells can comprise a nucleotide sequence that encodes a protein selected from the group consisting of a fluorescent protein, an enzyme that catalyzes production of a chromogenic or fluorescent product, an antigenic cell surface protein that is exposed to the cell exterior, or a fusion protein comprising at least one of the foregoing proteins, and the cells can then be purified by cell sorting, laser dissection, or immunoaffinity separation. The marker DNA construct can also comprise a nucleotide sequence encoding a protein that confers resistance to a selection agent, and purification of the cells can be performed by culturing the cells in the presence of an agent that selects against non-resistant cells. As described above, use of a bicistronic marker DNA construct such as a marker with cistrons or encoding GFP and Hygro^{r} would allows the identification of clones in which the marker is expressed by optical detection (of GFP fluorescence), and purification of the marked clones using a selecting agent (hygromycin) to selectively eliminate the unmarked cells.

Purification using a selectable marker is useful because it allows isolation of the marked cells without the need for trypsinization, which might destroy the diferentiation antigen, as discussed above.

The invention further provides methods wherein genetically unmodified stem cells are induced to differentiate into the particular differentiated or partially differentiated cell type having said differentiation antigen, and affinity methods employing antibodies that specifically bind to the differentiation antigen are used to isolate a purified population of differentiated, genetically unmodified cells having the differentiation antigen. A useful method comprises making antibodies against two differentiation antigens that are expressed at different stages of differentiation. For example, one differentiation antigen that is produced by cells at an early stage of differentiation, and another that is produced at a later stage of differentiation. Stem cells transfected with such a construct can be induced to differentiate, and a population of cells that has differentiated to the early stage of differentiation can be purified using antibodies against the first differentiation antigen. The cells can then be expanded and induced to differentiate further, and a population of cells that has differentiated to the later stage of differentiation can also be isolated using antibodies against the second differentiation antigen.

### Purified Partially or Fully Differentiated Cells

The present invention also provides compositions of purified populations of cells that have reached a particular stage of differentiation; for example, for producing and isolating different types of pluripotent stem cells, committed progenitor cells, or fully differentiated cells of a human or non-human mammal.

The present invention further provides compositions comprising purified, homogeneous populations of cells that are partially or fully differentiated into particular cell types that are the produced by the methods of the present invention, and can be used for basic biological and biomedical research, drug discovery and testing, toxicological testing and sensing, and for animal pre-testing and clinical testing of cell therapy methodologies.

### Using Purified, Differentiated Cell Types Prepared by the Present Invention

The present invention further provides methods for producing totipotent and pluripotent stem cells that are genetically selected and/or modified, for inducing such stem cells to partially or fully differentiate into particular cell types having the selected and/or modified genotypes, and for producing purified, homogeneous populations of such cells that can be used for basic biological and biomedical research, drug discovery and testing, toxicological testing and sensing, and for animal pre-testing and clinical testing of cell therapy methodologies.

### Uses of Purified Gene Trapped Cell Lines

Gene trapped lines can be purified as described above, e.g., by fluorescence sortiny, laser dissection, gene-trapped cell surface ligand expression and antibody mediated affinity removal, or by resistance to selection. Such defined and purified cell lines can then be used

to induce the differentiation through various kinds of cell-cell associations of other cell types into particularly types of differentiated cells; e.g., cardiac endothelial cells can be used to induce myocardial cell differentiation.

for drug discovery;

to prepare mRNA or proteins to induce more undifferentiated cells into the lineage defined by the gene-trapped lineage

for the generation of differentiation specific antibodies (such as injecting somatic cells into an animal to make a specific monoclonal or polyclonal antibody

for therapeutic testing in animals; and

actual cell therapeutics.

### An Improved Screen for Cell Inducers of Cell Differentiation

Isolated differentiated cells from the above techniques can be co-cultured (i.e., platal first under another cell, or on top, or simply in the same well showing the media) with another cell type. Various combinations of cell types can be screened to determine whether cell-cell induction altered the differentiated of the second cell type in an interesting and useful manner.

In another (and important) examples there are no assumptions made in regard to either the differentiated state of the inducer cell or the cell to be induced. Two cell populations (the “inducer cell” line and the “differentiated cell” line are co-cultured in a random manner to identify useful inducer-differentiated cell-cell interactions. An example of this invention is as follows: The inducer cell line is a characterized gene trapline that expresses a marker gene (GFP₁) when it differentiates into a particular types of cell. This cell line is co-cultured with a wide array of other distinct and characterized gene trap lines that when differentiated into a particular cell type express a distinct marker (GFP₂). These co-cultures are performed in many culture vessels (such as multiwell dishes) such that they can be screened for rare induction events. Evidence that one cell type induced the other would be a responsible juxtaposition of cells with each marker. This induction effect can then be verified by the isolation of differentiated animal marker expressing inducer cell and co-culturing the cells to be induced the differentiated target cells to a higher than background and change frequency of differentiation.

An important variation on the above technique would be to use a gene trap library with a single marker gene as both the inducer marker and the target differentiation marker. This would eliminate the need to generate redundant libraries. This inducer and differentiated cell lines can then be distinguished by the constitutive expression of exogenous marker such as β-gal, or by simple perfusion with the diffusible marker Di-I or similar markers.

The discovery of novel cell-cell induction pathways would greatly facilitate differentiation of particular useful cell types without growth modifications in many cases.

### Uses in Animal Pre-clinical Testing of Cell Thergpy, and for Actual in Cell Therapy

We have previously described types of therapies for which the cells can be used-pancreatic islet cells for diabetes, dopaminergic neurons for Parkinson's disease, myocardial cells for heart tissues, hepatocytes for liver disease, kidney cells for kidney tissue, hematopoietic stem cells for blood cell disorders and infectious disease, and vascular disorders, retinal cells for macular degeneration and other retinal diseases, chondrocytes for for arthritis, osteoblasts for bone diseases, myoblasts for muscle disorders, inner ear cells for hearing loss, etc.

### In Vitro use of Such Cells in Basic Research and Drug Discovery

In vitro cell uses include the use of purified differentiated cell types in cell screening to discover new therapeutics. An example would be isolated myocardial cells placed in a screen to identify agents that alter myocardial cell function. In addition such screens would be useful to test for toxicity of various agents. An example would be the isolation of human hepatocytes with normal P450 enzymes to perform toxicology studies. Another example would be intestinal epithelia to studying absorption of agents from the luminal to the basal aspect of such cells. The use of intestinal epithelial like cells is currently widespread in the industry and cultured conditions to model intestinal absorption is well-known in the current art. Currently intestinal epithelial-like cells are obtained from human cell lines such as Caco-2. However these cells are very abnormal. Intestinal epithelium made using the methods disclosed herein would more reliably reflect normal cell physiology. Such cells can also be coupled with devices and used to monitor for the presence of environmental toxins such and/or agents of biological warfare.

### Isolated Differentiated Cells are also Useful as Model Systems to Study Disease

### Secondary Modifications for Research and Drug Discovery

Once gene trapped animal stem cell lines are isolated that involve a differentiated cell type of interest from the perspective of cell biology and/or drug discovery (e.g., a myocardial cell), there cells can be isolated by using a co-expressed cell surface tag to which a specific antibody can react to isolate the cell of interested from a mixed population and such purified cell of interest can be used as in vitro model of disease (e.g., in the case of myocardial cell the beating heart muscle cells can be deprived of oxygen to mimic ischemia and/or exposed to candidate drugs to observe potential efficacy and to study potential toxicity.

In addition, genetic modifications can be introduced into these differentiated cells of interest, including targeted genetic modifications to introduce pathogenic mutations or to test potential therapeutic genetic modifications. Examples of such modification would be the introduction of the Alzheimer disease mutations and the subsequent differentiations of CNS neurons to model the disease in vitro, and/or text potential therapeutics in vitro.

Using nuclear transfer cloning, human stem cells can be generated that have a medically interesting phenotype. The method of the present invention can then be used to prepare purified populations of differentiated cells useful for medical research.

### Using Library of Gene Trap Cells for Making Homozygous Knockout Cells and Animals

Another example of a marker DNA construct that is useful in the present invention is a promoterless construct that includes sequences that mediate fusion of the marker mRNA to an mRNA encoded by one or more exons of the gene in which the marker DNA is inserted; e.g., a promoterless construct consisting of a splice acceptor site followed by a reporter gene and a poly-adenylation sequence (reviewed in Durick et al., Genome Res., 1999, 9:1019-25). Such a marker construct can be inserted into an intron so that it is transcriptionally regulated by the endogenous promoter, and its transcription produces a fusion mRNA formed by splicing of a splice donor sequence at the end of an endogenous exon with the splice acceptor sequence of the marker mRNA (see Durick et al.,1999).

Insertion of the above-described gene trap marker DNA constructs into the trapped gene usually prevents it from being expressed effectively, thereby “knocking out” the trapped gene; however, there are reports that such randomly inserted gene trap marker constructs can be co-expressed with the gene in which they are inserted without inhibiting the expression of the tagged gene (McClive et al., Dev. Dyn., 1998, 212(2):267-276; Voss et al., Dev. Dyn., 1998, 212(2):258-266; Voss et al., Dev. Dyn., 1998, 212(2):171-180).

While knocking out the expression of one allele of a genetic locus of a stem cell, referred to herein as a heterozygous knockout, frequently has no apparent effect on the differentiation of the stem cell, in some cases it can delay or change the course of differentiation of the stem cell and disrupt normal embryonic or fetal developement. As described below, gene trapped stem cells that are produced for the present invention in which a DNA marker construct is inserted to effect a heterozygous knockout can also be useful intermediates for producing stem cells in which both copies of the allele in the cell are knocked out, referred to herein as a homozygous knockout. For example, DNA of the gene in which the DNA marker construct is inserted can be cloned and used to disrupt the second copy of the allele in the stem cell line by homologous recombination. Stem cells with homozygous knockouts of genes can be used to produce differentiated cells or generate cloned animals that are of great value as experimental model systems for studying and testing drugs and other treatments for diseases associated with the mutation or loss of the genes.

Generation of gene trapped stem cells by randomly inserting promoterless marker DNA constructs and screening for cells containing differentiation-specific markers is an approach that is useful for large-scale, high-throughput screening to detect differentiationspecific genes. However, gene trapped stem cells useful for the present invention can also be made using homologous recombination to precisely insert a marker DNA construct into a selected site in a gene that is conditionally expressed in a differentiating cell. The DNA construct to be inserted can be designed to disrupt and inhibit expression of the endogenous gene, producing a heterozygous knockout; or it can be designed to be transcribed as a separate cistron without disrupting expression of the endogenous coding sequence, e.g., by including an IRES in the marker DNA construct and inserting it downstream of the endogenous stop codon (see Mountford et al., 1994).

Another use of the described technology stems from the fact that a gene trap will often reflect the insertion of the construct within the coding sequence of an allele of a gene.

Since one allele may be effectively knocked out by the insertion of the construct, if a chimeric animal such as a mouse mode from such cells, a homozygous knockout can easily be mode by breeding as is well described in the art.

This would allow both the isolation of such cells, but also the study of the function of the knockout in cell research and screening as just described, and may also have utility in cell therapy, as for example, the knockout of β2-microglobulin to improve the tolerance of allograft cells. Such double knockout cells can then be transfected or infected with another construct to make a new superimposed gene trap library and markers for various differentiating cell types observed for the second marker. This would allow the use of the knockout to be studied in any differentiated cell type isolated by gene trapping.

The library of gene trapped clones would be characterized as to what gene was trapped by sequencing away from the construct and getting a sequence tag that would then give you the gene by comparison with published databases. A customer who wants to know the effect of a knockout in an animal other than mouse (mouse knockouts easily done in other ways) the clone can be expanded and a knockout of the other allele performed (by homologous recombination) by us or the customer in many cases the customer will want us to perform both the knockout of the second allele, but also to differentiate the ES cell card to supply a specific differentiated cell type. An example would be the knockout of both alleles of the WRN (Wevner syndrome) gene to generate cells such as vascular endothelial cells that display accelerated cell senescence. By nuclear transfer, it is possible to take cells from another premature aging syndrome such as Hutchison Guilford syndrome (progeria) make ES cells and introduce the knockout of both alleles of WRN to obtain cells with both mutations.

Alternatively, the second allele (since it is the only one expressed) could also be modified or promoter or enhancer or other regulatory sequences modified to simply alter the only expressed alleles. Such modification can have use in understanding gene function, drug discovery, or in manufacturing cells that have therapeutic benefit.

### Business Strategy

The present invention makes possible a method of doing business comprising making a gene trap library of heterozygous knock-out ES cells, characterizing the knocked out genes, cataloging the heterozygous knock-out ES cells in the library, and advertising and offering to customers the service of preparing homozygous knock-out cells and cloned non-human animals to order, for drug discovery and drug testing.

### Use of ES Cells with Homogygous MHC Alleles

As described in earlier application purified cells made by the techniques described herein will be widely transplantable into individuals with genes with the homozygous alleles.

### Use of Purified Gene Trapped Cells for the Discovery of Cell Proliferation and Cell Survival Conditions

Such cells can be isolated as described above and screened against a panel of growth factors, ECM components, media, and other conditions that promotes their growth and/or survival. Such conditions would be useful in expanding these cells for banking, use in large-scale induction cDNA production, differentiation antigen identification etc.

### Compositions

The present invention further provides methods for making compositions that can be used in implementing the methods of the present invention.

### Oligonucleotides Complementary to Genes Expressed During Differentiation of a Particular Cell Type

The present invention also provides a composition comprising molecules and molecular arrays that can be used to detect the sequential expression of a set of genes that occurs in a cell lineage during differentiation of stem cells into a particular type. Examples of such molecules include nucleic acids that specifically hybridize to DNA sequences of genes that are expressed at different times in a cell of a lineage formed by differentiation of the stem cells into a particular type, or to the mRNA molecules encoded by such DNA sequences. Using known methods, such molecules can be used as hybridization probes or as amplification primers to detect the expression of genes in differentiating cells.

### Purified Partially or Fully Differentiated Cells

The present invention also provides compositions of pure populations of cells that have reached a particular stage of differentiation; for example, for producing and isolating different types of pluripotent stem cells, committed progenitor cells, or fully differentiated cells of a human or non-human mammal.

The present invention further provides compositions comprising purified, homogeneous populations of cells that are partially or fully differentiated into particular cell types that are the produced by the methods of the present invention, and can be used for basic biological and biomedical research, drug discovery and testing, toxicological testing and sensing, and for animal pre-testing and clinical testing of cell therapy methodologies.

### EXAMPLES

### Example I

### Primary Screening Ex Vivo

Promoterless DNA marker constructs encoding GFP are randomly inserted into the genomic DNA of a population of murine ES cells, and thousands of individual cells are isolated and expanded into lines of single-cell clones. Cells of different cloned ES cell lines are plated in triplicate in the wells of a 48-well tissue culture plates (1,000 ES cells per well; 16 different ES cell lines per plate), and the cells are cultured under conditions that induce differentiation. The cultured cells are continuously and automatically monitored to detect cells containing GFP for 12 weeks following the induction of differentiation—a time period sufficient to induce production of cells that have the appearance and biochemical markers of fully differentiated cells. Upon detection of GFP fluorescence in a well, a record is automatically made that identifies the ES cell line of the fluorescing cells, the time that GFP fluorescence was detected and the intensity of the signal. The temporal pattern of expression of GFP marker constructs in the cloned cells is shown in FIG. 2.

### Example 2

### Mapping a Branch of the Developmental Tree

A cloned gene trapped cell has a marker DNA construct encoding GFP inserted in gene A that is expressed when the cell is induced to differentiate. The node of the developmental tree corresponding to expression of gene A in the differentiating cell is shown in FIG. 3A.

To detect the conditional expression of additional genes in the lineage of gene A, a marker DNA construct encoding cyan fluorescent protein (CFP) is randomly inserted into the DNA of the population of cells with the gene A trap. The cells are then differentiated to obtain cells that express the marker for gene A, and these cells are screened to detect cells the appearance of a novel marker B; that is, to detect differentiating clones in which both A and B are expressed, with B following A temporally. Clone B is assigned to the missing region in the developmental tree as shown in FIG. 3B. The library of gene trapped stem cells expressing gene A is screened further to identify additional clones having marked genes that are expressed after gene A in the differentiating cell lineage. The cloning and screening steps can are then repeated—using stem cells in which genes A and B are trapped to make a new library of gene trapped cells, the cells are differentiated into the cell type of interest, and are screened to identify cells in which A and B plus a third marked gene are expressed in the differentiating clones. The method is repeated in this manner until all of the genes that are conditionally expressed in the lineage of cells differentiating into the cell type of interest are identified.

### Example 3

### Mapping the Differentiation of Human Cells into Cardiomyocytes

The following method is used to identify the nucleotide sequences and the temporal and cellular pattern of expression of genes that are transcriptionally activated in human cells that are differentiating into cardiomyocytes.

A. Analysis of Cardiomyocyte Differentiation in Cynomologous Monkeys:

A library of gene trapped stem cells is prepared using the totipotent stem cell line Cyno-1, which was isolated from the inner cell mass of parthenogenetic Cynomologous monkey embryos and is capable of differentiating into all the cell types of the body. (Cibelli et al., 2002, supra). Marker DNA constructs containing promoterless DNA cassettes encoding GFP are randomly inserted into the genomic DNA of a population of Cyno-1 cells, and clones having stably integrated constructs are subjected to primary screening ex vivo to identify cells having markers inserted into genes that are conditionally expressed in differentiating cells, as described above. Clones of gene trapped ES cells selected by primary screening are then cultured in vitro in the presence of dimethyl sulfoxide (DMSO) under conditions that induce differentiation of the stem cells to form cardiomyocytes (see Paquin et al., 2002, Proc. Natl. Acad. Sci., USA, 99:9550-9555), and the differentiating cells are monitored to detect expression of the marker genes. Marked genes that are conditionally expressed in differentiating cells are cloned and their nucleotide sequences are determined using known methods.

A secondary screening assay is performed by implanting the selected gene trapped ES cells into SCID mice to form teratomas, and these are examined histologically to detect and identify specific differentiated cell types. Differentiating or differentiated teratoma cells are examined by fluorescent microscopy to identify trapped genes that are conditionally expressed in these cells.

Nucleic acid hybridization probes complementary to DNA sequences of the conditionally expressed genes identified in the primary screening step are prepared and are labeled with fluorescent probes. In vivo secondary screening is performed by systematically examining developing Cynomologous embryos and fetuses histologically, identifying specific partially differentiated or fully differentiated cell types in the developing animals, and subjecting tissue sections containing these cells to FISH analysis, using the fluorescently labeled probes to detect the timing and location of transcriptional activation of the conditionally expressed genes in these cells.

Secondary screening in vivo is also performed by implanting Cyno-1 ES cells into SCID mice to form teratomas, examining these histologically to detect and identify specific differentiating or differentiated cell types, and analyzing the sections by FISH, using the above-described fluorescently labeled probes to detect expression in these cells of the conditionally expressed genes that were identified in the primary screening step.

The temporal and spatial patterns of the transcriptional activation of genes in cells giving rise to cardiomyocytes that are detected using the above-described secondary screening methods are analyzed, and a set of genes that are sequentially expressed in the lineage of cells giving rise to cardiomyocytes is identified. In addition, the timing of the transcriptional activation of each of these genes relative to the induction of differentiation, and the cell or tissue type in which the genes are expressed, are also determined.

B. Analysis of Cardiomyocyte Differentiation in Humans:

The information obtained by mapping a branch of the developmental tree of Cynomologous monkeys leading to the differentiation of cardiomyocytes as described above is used to obtain similar information for the differentiation of cardiomyocytes in a human being.

Human genes homologous to developmentally regulated genes expressed in cells of Cynomologous monkeys that are differentiating into cardiomyocytes are identified and cloned using known methods. The nucleotide sequences of monkey genes are compared to available databases of human genomic DNA sequences to identify the nucleotide sequences of the homologous human genes. Labeled nucleic acid hybridization probes complementary to the conditionally expressed monkey genes and to their human homologs are prepared and used to isolate the cloned human genes. Cloned DNAs containing the desired human homolog sequences are identified by their ability to hybridize specifically to both the monkey and human probes under high-stringency conditions. The human ES cells are then cultured in vitro in the presence of dimethyl sulfoxide (DMSO) under conditions that induce differentiation of the stem cells to form cardiomyocytes, and the cells are monitored by known methods to detect the timing of transcriptional activation of the homolog genes, and the cell types in which the expression occurs. For example, nucleic acid hybridization probes complementary to the human homolog DNA sequences are labeled with fluorescent probes, and the differentiating cells are assayed by FISH analysis, using the fluorescently labeled probes to detect the timing and cellular location of transcriptional activation of the conditionally expressed genes in the cultured cells.

Although the invention has been described in considerable detail with regard to certain embodiments thereof, it will be evident to those skilled in the art that other embodiments within the scope of the teachings of the present invention are possible. Accordingly, neither the disclosure nor the claims to follow are intended, nor should be construed to be, limited by the descriptions of the preferred embodiments disclosed here.

We claim:

1. A method for determining the relative timing of the transcriptional activation or repression of genes in a population of stem cells that occurs when the stem cells differentiate, comprising:
(a) randomly inserting into the genomic DNA of stem cells in a population of stem cells a marker DNA construct comprising a nucleotide sequence that encodes a detectable product and is not operably linked to a promoter;
(b) culturing the stem cells ex vivo under conditions in which the stem cells differentiate;
(c) monitoring the differentiating stem cells to detect changes in the level of expression of the marker DNA constructs in the cells; and
(d) detecting differentiating cells in which there is a change in the level of expression of the marker DNA construct, and determining the relative timing of the change in the level of expression of the marker DNA construct that occurs in these cells.

2. The method of claim 1, step further comprising isolating individual stem cells having an inserted marker DNA construct, or colonies of cloned cells derived from such individual stem cells; and then culturing the isolated stem cells in step (b).

3. The method of claim 1, wherein the stem cells are totipotent or nearly totipotent stem cells of a mammal.

4. The method of claim 1, wherein the stem cells embryonic stem cells of a murine, bovine, porcine, non-human primate, or human mammal.

5. The method of claim 1, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and a cell surface protein comprising an antigen that is exposed to the cell exterior.

6. The method of claim 1, further comprising identifying individual stem cells in which there is a change in the level of expression of the marker DNA construct when the stem cells differentiate, and isolating and expanding said stem cells as single cell clones.

7. The method of claim 6, further comprising determining the nucleotide sequence of at least part of the gene in which is inserted a marker DNA construct that changes its level of expression when a stem cell differentiates.

8. The method of claim 7, comprising determining the nucleotide sequence of at least part of the coding sequence or at least part of a transcription-regulating sequence of the gene.

9. The method of claim 1,
wherein the marker DNA construct encodes a recombinase,
wherein the same or a different marker DNA construct inserted into the genomic DNA of each stem cell comprises a first nucleotide sequence encoding a detectable product, and a second nucleotide sequence comprising two recombination sites that inhibits the expression of the first nucleotide sequence;
wherein the recombinase recognizes the two recombination sites, and expression of the nucleotide sequence encoding recombinase results in excision of the second nucleotide sequence from the stem cell genomic DNA and expression of the first nucleotide sequence encoding the detectable product; and
wherein step (d) comprises determining the relative timing of the transcriptional activation of the marker DNA constructs that occurs when the stem cells differentiate.

10. The method of claim 6, wherein the first nucleotide sequence encodes a protein selected from the group consisting of a fluorescent protein, an enzyme that catalyzes production of a chromogenic or fluorescent product, a protein that confers resistance to a selection agent, an intracellular antigenic protein, and a cell surface protein comprising an antigen that is exposed to the cell exterior.

11. The method of claim 2, further comprising
purifying differentiating cells having a transcriptionally activated marker DNA construct from cells that do not have a transcriptionally activated marker DNA construct, and
using the purified cells or an extract thereof as an immunogen to elicit production of an antibody that binds specifically to a differentiation antigen of the purified cells.

12. The method of claim 11, comprising eliciting production of an antibody that binds specifically to a differentiation antigen on the external surface of the cells.

13. The method of claim 12, comprising eliciting production of a monoclonal antibody that binds specifically to a differentiation antigen on the external surface of the cells.

14. The method of claim 11, wherein the marker DNA construct comprises a nucleotide sequence that encodes a protein selected from the group consisting of a fluorescent protein, an enzyme that catalyzes production of a chromogenic or fluorescent product, an antigenic cell surface protein that is exposed to the cell exterior, or a fusion protein comprising at least one of the foregoing proteins, and the cells are purified by cell sorting, laser dissection, or immunoaffinity separation.

15. The method of claim 11, wherein the marker DNA construct comprises a nucleotide sequence encoding a protein that confers resistance to a selection agent, and purification of the cells comprises culturing the cells in the presence of an agent that selects against non-resistant cells.

16. The method of claim 1 which includes a secondary screeding step to identify multiple genes that are transcriptionally activated in cells that differentiate into a particular cell phenotype.

17. The method claim 16 wherein screening is effected using an hyb probe, an antibody, or a 2-D gel.

18. A method for identifying genes in a cell that are transcriptionally activated at different times during differentiation of a stem cell into the particular cell type, comprising:
(a) identifying genes that are transcriptionally activated during differentiation of the stem cell into multiple cell types in vivo or ex vivo;
(b) inducing a population of said stem cells to differentiate into the particular cell type; and
(c) assaying to detect two or more of different genes identified in step
(a) that are transcriptionally activated at different times in cells that are differentiating into the particular cell type.

19. The method of claim 18, further comprising determining the nucleotide sequence of at least part of a gene detected in step (c) that is transcriptionally activated when the stem cells differentiate into the particular cell type.

20. The method of claim 18, wherein step (b) comprises assaying to detect a change in the concentration of a mRNA or a polypeptide resulting from transcriptional activation of a gene of the stem cell during differentiation of the stem cell into the particular cell type.

21. The method of claim 18, wherein the stem cell is a totipotent, nearly totipotent, or pluripotent stem cell.

22. The method of claim 18, wherein the stem cell is an embryonic stem cell or embryonic germ cell of a murine, bovine, porcine, non-human primate, or human mammal.

23. The method of claim 18, wherein at least one of the genes detected in step (c) is transcriptionally activated only in cells differentiating into the particular cell type.

24. The method of claim 18, wherein at least one of the genes detected in step (c) is transcriptionally activated in two or more lineages of cells differentiating into different cell types.

25. The method of claim 18, comprising inducing the stem cells to differentiate into a pluripotential stem cell or a progenitor cell committed to giving rise to one or a few fully differentiated cell types.

26. The method of claim 18, comprising inducing the stem cells to differentiate into a fully differentiated cell type.

27. The method of claim 18, comprising inducing the stem cells to differentiate into a particular cell type in vivo, and assaying to detect transcriptional activation of two or more genes at different times in vivo in cells differentiating into the particular cell type.

28. The method of claim 27, further comprising identifying the location in vivo of a cell in which is detected a gene that is transcriptionally activated in cells differentiating into the particular cell type.

29. The method of claim 27, further comprising determining the nucleotide sequence of at least part of a gene that is detected as being transcriptionally activated in cells differentiating into the particular cell type.

30. The method of claim 27, wherein assaying comprises monitoring to detect a change in the concentration of a mRNA encoded by one of the genes.

31. The method of claim 30, comprising monitoring to detect a change in the concentration of a mRNA by in situ hybridization with fluorescent hybridization probes (FISH) or radiolabeled hybridization probes.

32. The method of claim 30, comprising using reverse-transcription polymerase chain reaction (RT-PCR) to detect a change in the concentration of a mRNA.

33. The method of claim 18, wherein assaying comprises monitoring to detect a change in the concentration of a protein encoded by one of the genes.

34. The method of claim 33, wherein a protein encoded by one of the genes is an antigenic protein, and
the method comprises using antibodies that bind specifically to the antigenic protein in assaying to detect a change in the concentration of an antigenic protein.

35. The method of claim 27, wherein at least one marker DNA construct is inserted into the genomic DNA of the stem cells and is transcriptionally activated when the stem cells differentiate into the particular cell type; and the method comprises assaying to detect the expression of the marker DNA construct during the time the stem cells are differentiating into the particular cell type.

36. The method of claim 35 wherein the DNA construct is operably linked to an exogenous cell-specific promoter.

37. The method of claim 35 wherein the DNA construct is operably linked to an endogenous promoter.

38. The method of claim 35 wherein the cells are incorporated into a non-human animal and the assay is effected in vivo.

39. The method of claim 35, wherein the marker DNA construct is inserted into an endogenous gene and transcription of the marker DNA construct is controlled by the promoter of the endogenous gene.

40. The method of claim 39, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

41. The method of claim 27, wherein the stem cell is an embryonic stem cell, and the method comprises introducing the embryonic stem cell into a host nonhuman mammal in which it forms a teratoma comprising a differentiated cell of said particular cell type.

42. The method of claim 41 wherein said ES cells comprise a marker DNA construct randomly inserted that encodes a detectable product.

43. The method of claim 27 wherein the stem cell is a totipotent or nearly totipotent stem cell of a non-human mammal, the method further comprising:
generating a non-human embryo with the stem cell;
allowing the embryo to develop; and
assaying to detect transcriptional activation of two or more genes at different times in cells of the developing non-human mammal that are differentiating into the particular cell type.

44. The method of claim 43, wherein the stem cell is an embryonic stem cell or embryonic germ cell of a murine, bovine, porcine, or non-human primate mammal.

45. The method of claim 43 wherein said stem cells comprise a DNA encoding a detectable product randomly inserted into the genomic of said cells.

46. The method of claim 41, comprising generating a non-human embryo with the stem cell, in the genomic DNA of which is inserted a marker DNA construct that is transcriptionally activated in cells that are differentiating into the particular cell type;
wherein the assaying step comprises assaying to detect transcriptional activation of the marker DNA construct in cells that are differentiating into the particular cell type.

47. The method of claim 46 wherein the marker DNA is operably linked to an endogenous promoter or a heterologous promoter that is tissue specific.

48. The method of claim 46, wherein the marker DNA construct is inserted into an endogenous gene and transcription of the marker DNA construct is controlled by the promoter of the endogenous gene.

49. The method of claim 48, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

50. The method of claim 48, further comprising identifying the location in vivo of a cell having a gene that is transcriptionally activated in cells differentiating into the particular cell type.

51. The method of claim 48, wherein assaying comprises monitoring to detect a change in the concentration of a mRNA by in situ hybridization with fluorescent hybridization probes (FISH) or radiolabeled hybridization probes.

52. The method of claim 48, wherein assaying comprising using reverse-transcription polymerase chain reaction (RT-PCR) to detect a change in the concentration of a mRNA.

53. The method of claim 48, wherein a protein encoded by one of the genes is antigenic, and assaying comprises monitoring to detect a change in the concentration of an antigenic protein using antibodies that bind specifically to the antigenic protein.

54. The method of claim 53, comprising using immunocytochemistry to detect a change in the concentration of the antigenic protein.

55. The method of claim 48, further comprising determining the nucleotide sequence of at least part of a gene in which is inserted a marker DNA construct that is transcriptionally activated in cells that are differentiating into the particular cell type.

56. The method of claim 46, wherein the embryo is produced by nuclear transfer cloning using a nuclear donor cell in the genomic DNA of which is inserted a marker DNA construct that is transcriptionally activated when the stem cell differentiates into a particular cell type.

57. The method of claim 56 wherein the marker DNA operably linked to an endogenous or heterologous tissue specific promoter.

58. The method of claim 46, wherein the embryo is a chimeric embryo that comprises at least one stem cell that does not have said marker DNA construct.

59. The method of claim 58 wherein the marker DNA is operably linked to an endogenous or heterologous tissue specific promoter.

60. The method of claim 18, comprising inducing the stem cells to differentiate into the particular cell type ex vivo, and assaying to detect two or more genes that are transcriptionally activated at different times in cells of a lineage formed by differentiation of the stem cells into the particular cell type.

61. The method of claim 60, wherein the stem cells are embryonic stem cells or embryonic germ cells of a murine, bovine, porcine, non-human primate, or human mammal, and
the method comprises inducing the stem cells to form an embryoid body comprising a cell that differentiates into said particular cell type.

62. The method of claim 60, wherein step (b) comprises assaying to detect a change in the concentration of a mRNA or a polypeptide resulting from transcriptional activation of a gene of the stem cell during differentiation of the stem cell into the particular cell type.

63. The method of claim 60, wherein the stem cell is a totipotent, nearly totipotent, or pluripotent stem cell of a mammal.

64. The method of claim 60 wherein the cells are isolated or ______.

65. The method of claim 60 wherein the cells contain more than one marker gene.

66. The method of claim 60 wherein the sequence of the gene is determined a nucleic acid hybridization probe that hybridizes specifically to said nucleotide sequence of a gene that is transcriptionally activated when stem cells differentiates into a particular differentiated or partially differentiated cell type is used to identify said gene or related genes that are conditionally expressed in a particular cell type.

67. A method for identifying two or more genes that are transcriptionally activated at different times in cells that are differentiating into a particular cell type, comprising:
(a) obtaining stem cells comprising two or more different endogenous genes genes, in each of which is inserted a marker DNA construct that is controlled by the promoter of the endogenous gene and is transcriptionally activated when the stem cells differentiate;
(b) inducing the stem cells to differentiate into the particular cell type;
(c) monitoring to detect the activation of transcription of marker DNA constructs in the differentiating cells, and screening to identify two or more different genes containing marker DNA constructs that are transcriptionally activated at different times in cells differentiating into the particular cell type.

68. The method of claim 67, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

69. The method of claim 67, wherein monitoring comprises assaying to detect a change in the concentration of a mRNA or a polypeptide encoded by a marker DNA construct in cells differentiating into the particular cell type.

70. The method of claim 67 wherein the cells contain more than one marker gene.

71. The method of claim 67 wherein the marker gene is operably linked to an endogenous or tissue specific promoter.

72. The method of claim 67, wherein at least one of the genes detected in step (c) is transcriptionally activated only in cells differentiating into the particular cell type.

73. The method of claim 67, wherein at least one of the genes detected in step (c) is transcriptionally activated in two or more lineages of cells differentiating into different cell types

74. The method of claim 67, further comprising determining the nucleotide sequence of at least part of an endogenous gene in which is inserted a marker DNA construct that is transcriptionally activated in cells that are differentiating into the particular cell type.

75. The method of claim 67, comprising inducing the stem cells to differentiate into a pluripotential stem cell or a progenitor cell committed to giving rise to one or a few fully differentiated cell types.

76. The method of claim 67, comprising inducing the stem cells to differentiate into a fully differentiated cell type.

77. The method of claim 71 wherein one marker is recombinant mediated and the other marker is randomly inserted.

78. The method of claim 67, comprising inducing the stem cells to differentiate into the particular cell type in vivo, and monitoring to detect the activation of transcription of marker DNA constructs in cells that are differentiating in vivo into the particular cell type.

79. The method of claim 78, further comprising identifying the location in vivo of a cell containing a marker DNA construct that is transcriptionally activated in cells differentiating into the particular cell type.

80. The method of claim 77, wherein assaying comprises monitoring to detect a change in the concentration of a mRNA encoded by a marker DNA construct by in situ hybridization with fluorescent hybridization probes (FISH) or radiolabeled hybridization probes.

81. The method of claim 77 wherein marker DNA constructs encode an antigenic protein, and assaying comprises monitoring to detect a change in the concentration of the antigenic protein using antibodies that bind specifically to the antigenic protein.

82. The method of claim 81, comprising using immunocytochemistry to detect a change in the concentration of the antigenic protein.

83. The method of claim 77, further comprising determining the nucleotide sequence of at least part of an endogenous gene in which is inserted a marker DNA construct that is transcriptionally activated in cells that are differentiating in vivo into the particular cell type.

84. The method of claim 77, wherein the stem cell is a totipotent or nearly totipotent stem cell of a non-human mammal, the method further comprising:
generating a non-human embryo with the stem cell;
allowing the embryo to develop; and
monitoring to detect the activation of transcription of the marker DNA constructs in cells of the developing non-human mammal that are differentiating into the particular cell type.

85. The method of claim 84, wherein the stem cell is an embryonic stem cell or embryonic germ cell of a murine, bovine, porcine, or non-human primate mammal.

86. The method of claim 84, further comprising identifying the location in vivo of a cell containing a marker DNA construct that is transcriptionally activated in cells differentiating into the particular cell type.

87. The method of claim 84, wherein the embryo is produced by nuclear transfer cloning using a nuclear donor cell in the genomic DNA of which is inserted a marker DNA construct that is transcriptionally activated when the stem cell differentiates into a particular cell type.

88. The method of claim 84, wherein the embryo is a chimeric embryo that comprises at least one stem cell that does not have said marker DNA construct.

89. The method of claim 77, wherein the stem cell is an embryonic stem cell, and the method further comprises:
introducing the embryonic stem cell into a host non-human mammal in which it forms a teratoma comprising a differentiated cell of the particular cell type; and
monitoring to detect the activation of transcription of the marker DNA constructs in cells of the teratoma that are differentiating into the particular cell type.

90. The method of claim 67, further comprising purifying a cell containing a marker DNA construct that is transcriptionally activated in cells differentiating into the particular cell type by separating the cell from cells in which said marker DNA construct is not transcriptionally activated.

91. The method of claim 90, wherein the marker DNA construct encodes an antigenic cell surface protein, and the cells having the antigenic cell surface protein are purified from cells that do not have the protein by laser dissection, cell sorting, or immunoaffinity separation.

92. The method of claim 90, wherein the marker DNA construct encodes a protein selected from the group consisting of a fluorescent protein, an enzyme that catalyzes production of a chromogenic or fluorescent product, an antigenic cell surface protein that is exposed to the cell exterior, or a fusion protein comprising at least one of the foregoing proteins, and the cells are purified by cell sorting, laser dissection, or immunoaffinity separation.

93. The method of claim 90, wherein the marker DNA construct encodes a protein that confers resistance to a selection agent, and purification of the cells comprises culturing the cells in the presence of an agent that selects against non-resistant cells.

94. The method of claim 67, wherein step (a) comprises obtaining multiple lines of a stem cell, each line having at least one gene in which is inserted a marker DNA construct that is transcriptionally activated when the stem cells differentiate;
and the method further comprises identifying cells of two or more different lines of the stem cell, each having a different gene in which is inserted a marker DNA construct that is transcriptionally activated at a different time in cells differentiating into the particular cell type.

95. The method of claim 94 wherein marker is operably linked to an endogenous or heterologous tissue specific promoter.

96. The method of claim 68, wherein step (a) comprises obtaining multiple lines of a stem cell, each line having two or more genes in which are inserted marker DNA constructs that are transcriptionally activated when the stem cells differentiate;
and the method further comprises identifying at least one stem cell line having two or more genes in which are inserted marker DNA constructs that are transcriptionally activated at different times in cells differentiating into the particular cell type.

97. The method of claim 1, wherein
step (a) comprises randomly inserting into at least two different sites in the genomic DNA of each stem cell in a population of stem cells,
wherein each inserted marker DNA construct comprises a nucleotide sequence that encodes the same or a different detectable product, at least one of which nucleotide sequences is not operably linked to a promoter; and
step (c) comprises monitoring the differentiating stem cells to detect changes in the level of expression of both marker DNA constructs in each stem cell.

98. The method of claim 97, wherein at least two marker DNA constructs inserted in the genomic DNA of each stem cell encode different detectable products.

99. The method of claim 97, wherein at least one marker DNA construct in each stem cell comprises an exogenous promoter that is operably linked to a nucleotide sequence that encodes a detectable product and is transcriptionally activated in cells that are differentiating into a particular cell type.

100. The method of claim 97, further comprising identifying individual stem cells having at least two marker DNA constructs that are transcriptionally activated or repressed when the stem cells differentiate, and isolating and expanding said stem cells as single cell clones.

101. The method of claim 1,
wherein the marker DNA construct encodes a recombinase,
wherein the same or a different marker DNA construct inserted into the genomic DNA of each stem cell comprises a first nucleotide sequence encoding a detectable product, and a second nucleotide sequence comprising two recombination sites that inhibits the expression of the first nucleotide sequence;
wherein the recombinase recognizes the two recombination sites, and expression of the nucleotide sequence encoding recombinase results in excision of the second nucleotide sequence from the stem cell genomic DNA and expression of the first nucleotide sequence encoding the detectable product; and
wherein step (d) comprises determining the relative timing of the transcriptional activation of the marker DNA constructs that occurs when the stem cells differentiate.

102. The method of claim 1,
wherein the marker DNA construct encodes a recombinase,
wherein the same or a different marker DNA construct inserted into the genomic DNA of each stem cell comprises:
(i) a first nucleotide sequence encoding a detectable product, and
(ii) a second nucleotide sequence comprising two recombination sites that inhibits the expression of the first nucleotide sequence;
wherein the recombinase recognizes the two recombination sites, and expression of the nucleotide sequence encoding recombinase results in excision of the second nucleotide sequence from the stem cell genomic DNA and expression of the first nucleotide sequence encoding the detectable product.

103. A composition comprising isolated stem cells comprising two or more marker DNA constructs that are transcriptionally activated at different times during differentiation of the stem cells into a particular cell type.

104. The composition of claim 103 wherein the marker genes are operably linked to tissue specific heterologous promoters.

105. The composition of claim 103, wherein at least one marker DNA construct is inserted into an endogenous gene and its transcription is controlled by the endogenous promoter of the gene in which it is inserted.

106. The composition of claim 103, wherein transcription of at least one of the marker DNA constructs is controlled by an exogenous promoter that is a cell type-specific or differentiation stage-specific promoter.

107. The composition of claim 103 comprising cells of two or more different lines of said stem cell, the cells of each line having at least one gene in which is inserted a marker DNA construct that is transcriptionally activated at a different time interval during differentiation of the stem cells into the particular cell type.

108. The composition of claim 103 comprising at least one stem cell having two or more different genes, in each of which is inserted a marker DNA construct that is transcriptionally activated at a different time during differentiation of the stem cell into the particular cell type.

109. The composition of claim 103, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

110. The composition of claim 103, wherein the stem cell is an embryonic stem cell or embryonic germ cell of a murine, bovine, porcine, non-human primate, or human mammal.

111. A method for detennining conditions that induce stem cells to differentiate or partially differentiate in vitro into a particular cell type, comprising,
(a) obtaining stem cells having genes in which a marker DNA construct is randomly inserted,
(b) screening to identify stem cells comprising two or more different genes in which are inserted marker DNA constructs that are transcriptionally activated at different times in cells of the lineage formed by differentiation of the stem cells into the particular cell type;
(c) culturing the stem cells identified in (b) in the presence of one or more combinations of chemical, biological, and physical agents that are putatively differentiation-inducing, and assaying to identify one or more combinations of conditions in which the two or more genes are transcriptionally activated; and
(d) culturing the stem cells under the conditions identified in step (c) to induce the stem cells to differentiate or partially differentiate into the particular cell type.

112. The method of claim 111, further comprising isolating the stem cells identified in step (b), and expanding the isolated stem cells as single cell clones.

113. The method of claim 111, wherein the stem cells are totipotent or nearly totipotent stem cells.

114. The method of claim 111, wherein the stem cells are embryonic stem cells or embryonic germ cells of a murine, bovine, porcine, non-human primate, or human mammal.

115. The method of claim 111, wherein the marker DNA construct encodes a protein selected from the group consisting of a fluorescent protein, an enzyme that catalyzes production of a chromogenic or fluorescent product, a protein that confers resistance to a selection agent, an intracellular antigenic protein, and an antigenic cell surface protein that is exposed to the cell exterior.

116. The method of claim 111, further comprising determining the nucleotide sequence of at least part of the gene in which is inserted a marker DNA construct that is transcriptionally activated when the stem cell differentiates.

117. The method of claim 111, wherein step (c) comprises assaying to detect a change in the concentration of a mRNA resulting from transcriptional activation of a gene in a cell of the lineage formed by differentiation of said stem cell into said particular cell type.

118. The method of claim 117, comprising assaying to detect a change in the concentration of a mRNA by in situ hybridization with fluorescent hybridization probes (FISH) or radiolabeled hybridization probes.

119. The method of claim 118, comprising using reverse-transcription polymerase chain reaction (RT-PCR) to detect a change in the concentration of a mRNA.

120. The method of claim 111, wherein step (c) comprises assaying to detect a change in the concentration of a protein that is synthesized during differentiation of said stem cell into a particular cell type.

121. The method of claim 118, comprising assaying to detect a change in the concentration of a surface protein comprising an antigenic portion that is exposed to the cell exterior.

122. The method of claim 111, wherein at least one of said two or more genes identified in step (b) is a uninodal locus that is only transcriptionally activated during differentiation of the stem cell into the particular cell type.

123. The method of claim 111, wherein at least one of said two or more genes identified in step (b) is a multinodal locus that is transcriptionally activated when the stem cell differentiates into at least two different cell types.

124. A method for inducing stem cells to differentiate or partially differentiate in vitro into a particular cell type, comprising,
(a) identifying a first gene that is transcriptionally activated in a cell of the lineage formed by differentiation of the stem cells into the particular cell type;
(b) identifying a second gene that is transcriptionally activated after the first gene in a cell of the lineage formed by differentiation of the stem cells into the particular cell type;
(c) culturing the stem cells in the presence of at least one combination of chemical, biological, and physical agents in which the first gene is transcriptionally activated,
(d) purifying the partially differentiated cells by separating cells in which the first gene is transcriptionally activated from cells in which it is not transcriptionally activated; and
(e) culturing the stem cells in the presence of at least one combination of chemical, biological, and physical agents in which the second gene is transcriptionally activated, to induce the stem cells to further differentiate or partially or differentiate into the particular cell type.

125. The method of claim 124, wherein the second gene is transcriptionally activated in cells that are differentiated into the particular cell type.

126. The method of claim 124, wherein the combination of chemical, biological, and physical agents in step (c) is different from the combination of chemical, biological, and physical agents in step (e).

127. The method of claim 124, wherein the first gene encodes an antigenic cell surface protein that is exposed to the cell exterior, and the cells having the antigenic cell surface protein are purified from cells that do not have the protein.

128. A composition comprising two stem cell lines, each having a separate marker of differentiation

129. A method for identifying cell-cell interactions that induce differentiation, comprising screening sets of at least two cell lines, each having a separate marker of differentiation, in order to identify cell pairs that interact based on the expression of a differentiated marker.

130. A method for inducing stem cells to differentiate or partially differentiate in vitro into a particular cell type, comprising,
(a) identifying two or more genes that are transcriptionally activated at different times in a cell of the lineage formed by differentiation of the stem cells into the particular cell type;
(b) culturing the stem cells in the presence of one or more combinations of chemical, biological, and physical agents in which the two or more genes identified in step (a) are sequentially transcriptionally activated, to induce the stem cells to differentiate or partially differentiate into the particular cell type.

131. The method of claim 130, wherein step (a) comprises
obtaining multiple lines of a stem cell, each line having at least one gene in which is inserted a marker DNA construct that is transcriptionally activated when the stem cells differentiate; and
identifying cells of two or more different lines of the stem cell, each having a different gene in which is inserted a marker DNA construct that is transcriptionally activated at a different time in a cell of a lineage formed by differentiation of the stem cell into the particular cell type;
and step (b) comprises culturing the stem cells identified in step (a) in the presence of the differentiation-inducing agents to induce the stem cells to differentiate or partially differentiate into the particular cell type.

132. The method of claim 130, wherein step (a) comprises
obtaining multiple lines of a stem cell, each line having two or more genes in which are inserted marker DNA constructs that are transcriptionally activated when the stem cells differentiate;
identifying at least one stem cell line having two or more genes in which are inserted marker DNA constructs that are transcriptionally activated at different times in a cell of a lineage formed by differentiation of the stem cell into the particular cell type;
and step (b) comprises comprises culturing the stem cells identified in step (a) in the presence of the differentiation-inducing agents to induce the stem cells to differentiate or partially differentiate into the particular cell type.

133. The method of claim 130, wherein the stem cell is a totipotent or nearly totipotent stem cell.

134. The method of claim 130, wherein the stem cell is an embryonic stem cell or embryonic germ cell of a murine, bovine, porcine, non-human primate, or human mammal.

135. The method of claim 130, further comprising purifying the differentiated or partially differentiated cells by separating cells in which at least one of the genes identified in step (a) is transcriptionally activated from cells in which said locus is not transcriptionally activated.

136. The method of claim 129, wherein at least one of the genes identified in step (a) encodes an antigenic cell surface protein that is exposed to the cell exterior, and the cells having the antigenic cell surface protein are purified from cells that do not have the protein.

137. The method of claim 130, wherein the cells having the antigenic cell surface protein are purified from cells that do not have the protein by laser dissection, cell sorting, or immunoaffinity separation.

138. The method of claim 135,
wherein the stem cells have inserted into their genome a marker DNA construct comprising a nucleotide sequence encoding a polypeptide that is transcriptionally activated when at least one of the genes identified in step (a) is transcriptionally activated,
and the differentiated or partially differentiated cells are purified by separating cells in which the marker DNA construct is expressed from cells in which it is not expressed.

139. The method of claim 138, wherein the marker DNA construct comprises a nucleotide sequence that encodes a protein selected from the group consisting of a fluorescent protein, an enzyme that catalyzes production of a chromogenic or fluorescent product, an antigenic cell surface protein that is exposed to the cell exterior, or a fusion protein comprising at least one of the foregoing proteins, and the cells are purified by cell sorting, laser dissection, or immunoaffinity separation.

140. The method of claim 138, wherein the marker DNA construct comprises a nucleotide sequence encoding a protein that confers resistance to a selection agent, and purification of the cells comprises culturing the cells in the presence of an agent that selects against non-resistant cells.

141. The method of claim 138, wherein the marker DNA construct comprises at least two nucleotide sequences that each encode a polypeptide and are expressed in a cell of the lineage formed by differentiation of the stem cell into the particular cell type.

142. The method of claim 138, wherein the marker DNA construct comprises a nucleotide sequence that is operably linked to promoter that is of the same or different species as the stem cells, and is a constitutive promoter, an inducible promoter, a developmental stage-specific promoter, or a cell type-specific promoter.

143. The method of claim 138,
wherein a marker DNA construct is inserted into at least one of the genes identified in step (a) and is transcriptionally activated when the locus in which it is inserted is transcriptionally activated,
and the differentiated or partially differentiated cells are purified by separating cells in which the marker DNA construct is expressed from cells in which it is not expressed.

144. The method of claim 143, wherein insertion of the marker DNA construct into a gene identified in step (a) partially or completely inhibits the expression of the protein encoded by the gene.

145. The method of claim 143, wherein insertion of the marker DNA construct into a gene identified in step (a) of a stem cell does not inhibit the expression of the protein encoded by the gene.

146. The method of claim 138,
wherein the marker DNA construct comprises a nucleotide sequence encoding a detectable product operably linked to an conditional promoter that activates transcription of the marker DNA at approximately the same time that at least one of the genes identified in step (a) is transcriptionally activated;
and the partially or completely differentiated cells are purified by separating cells in which the marker DNA construct is expressed from cells in which it is not expressed.

147. The method of claim 146, wherein the conditional promoter is an inducible promoter, a developmental stage-specific promoter, or a cell type-specific promoter, and is of the same or different species as the stem cells.

148. The method of claim 138,
wherein the marker DNA construct is inserted into at least one of the genes identified in step (a) and comprises a nucleotide sequence encoding a recombinase that is transcriptionally activated when the locus in which it is inserted is transcriptionally activated;
and the same or a different marker DNA construct inserted in the genome of the stem cells also comprises a first nucleotide sequence encoding a detectable product, and a second nucleotide sequence comprising two recombination sites that inhibits the expression of the first nucleotide sequence; and
wherein the recombinase recognizes the two recombination sites, and expression of the nucleotide sequence encoding recombinase results in excision of the second nucleotide sequence from the stem cell genomic DNA and expression of the first nucleotide sequence encoding the detectable product.

149. The method of claim 138,
wherein the marker DNA construct comprises a nucleotide sequence encoding a detectable product, and further comprises two recombination sites flanking at least one nucleotide sequence that is required for expression of the first DNA;
and the same or a different marker DNA construct inserted in the genome of the stem cells comprises a conditional promoter that is operably linked to an nucleotide sequence encoding a recombinase that recognizes the two recombination sites; and
wherein activation of transcription of the nucleotide sequence encoding the recombinase results in excision of the nucleotide sequence required for expression of the DNA encoding the detectable product, thereby inhibiting expression of the DNA encoding the detectable product.

150. The method of claim 149, wherein the conditional promoter is of the same or different species as the stem cells, and is a constitutive promoter, an inducible promoter, a developmental stage-specific promoter, or a cell type-specific promoter.

151. A method for producing an antibody that binds specifically to a differentiation antigen of a particular differentiated or partially differentiated cell type, comprising,
(a) obtaining a stem cell having inserted in its genomic DNA a marker DNA construct comprising a nucleotide sequence encoding a detectable product that is transcriptionally activated in cells of a particular differentiated or partially differentiated cell type,
(b) inducing the stem cells to differentiate into the particular differentiated or partially differentiated cell type;
(c) monitoring the differentiating stem cells to detect activation of transcription of the marker DNA construct in the cells;
(d) purifying cells having a transcriptionally activated marker DNA construct; and
(e) using the purified cells or an extract thereof as an immunogen to elicit production of an antibody that binds specifically to a differentiation antigen of the purified cells.

152. The method of claim 151, wherein step (b) comprises culturing the cells ex vivo under conditions in which the stem cells differentiate into the particular differentiated or partially differentiated cell type.

153. The method of claim 151, wherein step (e) comprises eliciting production of an antibody that binds specifically to a differentiation antigen on the external surface of the cells.

154. The method of claim 153, comprising eliciting production of a monoclonal antibody that binds specifically to a differentiation antigen on the external surface of the cells.

155. The method of claim 153, comprising eliciting production of polyclonal antibodies that bind specifically to a differentiation antigen on the external surface of the cells.

156. The method of claim 152, wherein the marker DNA construct comprises a nucleotide sequence that encodes a protein selected from the group consisting of a fluorescent protein, an enzyme that catalyzes production of a chromogenic or fluorescent product, an antigenic cell surface protein that is exposed to the cell exterior, or a fusion protein comprising at least one of the foregoing proteins, and the cells are purified by cell sorting, laser dissection, or immunoaffinity separation.

157. The method of claim 152, wherein the marker DNA construct comprises a nucleotide sequence encoding a protein that confers resistance to a selection agent, and purification of the cells comprises culturing the cells in the presence of an agent that selects against non-resistant cells.
